SEQUENCE LISTING (19 sequences)

SEQ ID NO: 1 - protein, 275 aa, Rattus norvegicus
MTPRAQLLPLLLATYTVVAAAVTSDEPTKTLSPATGDATLAFVFDVTGSMWDDLMQVIDGASRILERSLSSRSRVIANYALVPFHDPDIGPVTLTADPVVFQRELRQLYVQGGGDCPEMSVGAIKAAVEVANPGSFIYVFSDARAKDYHKKNELLQLLQLKQSQVVFVLTGDCGDRTHPGYLAFEEIASTSSGQVFQLDKQQVSEVLKWVESAIQASKVHLLSADHEEEGEHTWRIPFDPSLKEVTISLSGPGPEIEVRDPLGMSQGSPPLLMQD

SEQ ID NO: 2 - DNA, 1031 bp, Rattus norvegicus
  CDS (96)...(920)
tctagcgaaccccttcggcccgctagagcgagactgcactgccatctatccctgcgacctgcgcgtcccattagggctgcagcctccggctcagcatgacgcctagggcgcagctcctgccgctgctcctggcgacctacacagtagtggcggcggcggtcacatctgatgagcccacgaagacgctgtcccccgccacaggagacgccaccctggccttcgtcttcgatgtcaccggctccatgtgggacgatctgatgcaggtgatcgacggcgcctcacgcattctggagcgcagtctgagcagccgcagccgggtcatcgccaactatgcgctggtgcctttccacgacccagacattggcccagtgaccctcacggcggacccagtggtgtttcagagagagctgagacaactctatgttcagggaggtggtgactgcccagaaatgagtgtgggggccatcaaggctgccgtggaggttgccaaccccggctccttcatctacgtcttctcggatgcccgtgccaaggactaccacaagaagaatgagctcctgcagctcctgcagctgaagcagtcgcaggtggtcttcgtgctgactggggactgcggtgaccgcacccaccctggctacctggcttttgaggagatcgcctccaccagttctggccaagtgttccagctggacaagcagcaggtgtcggaggtgttaaagtgggtggagtccgccatccaggcctccaaagttcatctgctgtcagcagaccacgaggaggagggcgaacacacatggagaatcccttttgaccccagcttgaaggaagtcaccatctcactgagcgggccagggcctgagatcgaagtccgggacccactgggtatgtcccagggttcacctcctcttctgatgcaagactgagctggaaggccaggctgaggcgatggaaggaggggcctgaggagatggctcagccaataaaatgtctgcctcacacaaaaaaaaaaaaagcccggctcgagcggccgc

SEQ ID NO: 3 - DNA, 25 bp, Artificial Sequence
  synthetic
cgtatgttgtgtggaattgtgagcg

SEQ ID NO: 4 - DNA, 25 bp, Artificial Sequence
  synthetic
gatgtgctgcaaggcgattaagttg

SEQ ID NO: 5 - DNA, 28 bp, Artificial Sequence
  synthetic
gccgccagtgtgctggaattcggctagc

SEQ ID NO: 6 - DNA, 28 bp, Artificial Sequence
  synthetic
cgaattctgcagatatccatcacactgg

SEQ ID NO: 7 - DNA, 25 bp, Artificial Sequence
  synthetic
ctagagggcccaattcgccctatag

SEQ ID NO: 8 - DNA, 25 bp, Artificial Sequence
  synthetic
tgagtcgtattacaattcactggcc

SEQ ID NO: 9 - DNA, 20 bp, Artificial Sequence
  synthetic
gctcggatccactagtaacg

SEQ ID NO: 10 - DNA, 18 bp, Artificial Sequence
  synthetic
tttttttttttttttttt

SEQ ID NO: 11 - DNA, 25 bp, Artificial Sequence
  synthetic
cgtatgttgtgtggaattgtgagcg

SEQ ID NO: 12 - DNA, 25 bp, Artificial Sequence
  synthetic
gatgtgctgcaaggcgattaagttg

SEQ ID NO: 13 - DNA, 20 bp, Artificial Sequence
  synthetic
tggccttcgtcttcgatgtc

SEQ ID NO: 14 - DNA, 18 bp, Artificial Sequence
  synthetic
gccgtcgatcacctgcat

SEQ ID NO: 15 - DNA, 22 bp, Artificial Sequence
  synthetic
ccggctccatgtgggacgatct

SEQ ID NO: 16 - DNA, 20 bp, Artificial Sequence
  synthetic
cggctaccacatccaaggaa

SEQ ID NO: 17 - DNA, 18 bp, Artificial Sequence
  synthetic
gctggaattaccgcggct

SEQ ID NO: 18 - DNA, 22 bp, Artificial Sequence
  synthetic
tgctggcaccagacttgccctc

SEQ ID NO: 19 - DNA, 1031 bp, Rattus norvegicus
agatcgcttggggaagccgggcgatctcgctctgacgtgacggtagatagggacgctggacgcgcagggtaatcccgacgtcggaggccgagtcgtactgcggatcccgcgtcgaggacggcgacgaggaccgctggatgtgtcatcaccgccgccgccagtgtagactactcgggtgcttctgcgacagggggcggtgtcctctgcggtgggaccggaagcagaagctacagtggccgaggtacaccctgctagactacgtccactagctgccgcggagtgcgtaagacctcgcgtcagactcgtcggcgtcggcccagtagcggttgatacgcgaccacggaaaggtgctgggtctgtaaccgggtcactgggagtgccgcctgggtcaccacaaagtctctctcgactctgttgagatacaagtccctccaccactgacgggtctttactcacacccccggtagttccgacggcacctccaacggttggggccgaggaagtagatgcagaagagcctacgggcacggttcctgatggtgttcttcttactcgaggacgtcgaggacgtcgacttcgtcagcgtccaccagaagcacgactgacccctgacgccactggcgtgggtgggaccgatggaccgaaaactcctctagcggaggtggtcaagaccggttcacaaggtcgacctgttcgtcgtccacagcctccacaatttcacccacctcaggcggtaggtccggaggtttcaagtagacgacagtcgtctggtgctcctcctcccgcttgtgtgtacctcttagggaaaactggggtcgaacttccttcagtggtagagtgactcgcccggtcccggactctagcttcaggccctgggtgacccatacagggtcccaagtggaggagaagactacgttctgactcgaccttccggtccgactccgctaccttcctccccggactcctctaccgagtcggttattttacagacggagtgtgtttttttttttttcgggccgagctcgccggcg

(19) United States
(12) Patent Application Publication
(10) Pub. No.: US 2005/0003364 A1
(43) Pub. Date: Jan. 6, 2005

(54) Secreted factors

(76) Inventors: Lawrence W. Stanton, Redwood City, CA (US); Ann M. Kapoun, Palo Alto, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 10/638,141

(22) Filed: Aug. 7, 2003

Related U.S. Application Data
(62) Division of application No. 09/665,728, filed on Sep. 20, 2000.
(60) Provisional application No. 60/193,548, filed on Mar. 31, 2000.

(51) Int. Cl. C12Q 1/68

(52) U.S. Cl. 435/6

(57) ABSTRACT

The invention concerns a new secreted factor encoded by clone P00210_D09 and other mammalian homologues and variants of such factor, as well as polynucleotides encoding them. The invention further concerns methods and means for producing such factors and their use in the diagnosis and treatment of various cardiac, renal or inflammatory diseases.

This application is a divisional application of U.S. patent Ser. No. 09/665,728, which claims benefit under Title 35, United States Code § 119(e) of U.S. provisional application No. 60/193,548 filed on Mar. 31, 2000; both of which are hereby incorporated by reference in their entirety.

### FIELD OF THE INVENTION

The present invention concerns secreted factors encoded by genes differentially regulated in certain diseased tissues. More particularly, the invention concerns nucleic acid encoding novel secreted polypeptide factors, the encoded polypeptides, and compositions containing and methods and means for producing them. The invention further concerns methods based on the use of such nucleic acids and/or polypeptides in the diagnosis and treatment of various diseases, in particular cardiac, renal, or inflammatory diseases.

### BACKGROUND OF THE INVENTION

Gene expression patterns, including changes in gene expression between normal and diseased tissues or tissues in various stages of disease progression provide valuable insight into the molecular determinants of normal and abnormal cellular physiology. Accordingly, genes that are differentially expressed in subjects suffering from a disease, such as cardiac, renal or inflammatory disease, relative to normal subjects, are useful targets for intervention to diagnose, prevent or treat such diseases.

Techniques have been developed to efficiently analyze the level of expression of specific genes in cells and tissues. Procedures that can be used to identify and clone differentially expressed genes include, for example, subtractive hybridization (Jiang and Fisher, Mol. Cell. Different. 1:285-299 [1993]; Jiang et al., Oncogene 10, 1855-1864 [1995]; Sagerstrom et al., Annu. Rev. Biochem. 66: 751-783 [1997]); differential RNA display (DDRT-PCR) (Watson et al., Developmental Neuroscience 15:77-86 [1993]; Liang and Pardee, Science 257:967-971 [1992]); RNA fingerprinting by arbitrarily primed PCR (RAP-PCR) (Ralph et al., Proc. Natl. Acad. Sci. USA 90:10710-10714 [1993]; McClelland and Welsh, PCR Methods and Applications 4:S66-81 [1994]); representational difference analysis (RDA) (Hubank and Schatz, Nucl. Acids Res. 22:5640-5648 [1994]); serial analysis of gene expression (SAGE) (Velculescu et al., Science 270:484-487 [1995]; Zhang et al., Science 276:1268-1272 [1997]); electronic subtraction (Wan et al., Nature Biotechnology 14:1685-1691 [1996]); combinatorial gene matrix analyses (Schena et al., Science 270:467-470 [1995]), and various modifications and improvements of these and similar techniques.

A particularly attractive method for assessing gene expression is the DNA microarray technique. In this method, nucleotide sequences of interest are plated, or arrayed, on a porous or non-porous substrate that can be paper, nylon or other type of membrane, filter, chip, glass slide or any other suitable solid support. The arrayed sequences are then hybridized with specific DNA probes from cells or tissues of interest. Microarrays of biological materials have been described in a number of patents and patent applications, including, for example, U.S. Pat. Nos. 5,744,305; 5,800, 992; 5,807,522; 5,716,785; and European Patent No. 0 373 203.

The DNA microarray technique can be used to monitor the expression level of large numbers of genes simultaneously (to produce a transcript image), and to identify genetic variants, mutations and polymorphisms. This information may be used to determine gene function, understanding the genetic basis of disease, diagnosing disease, and developing and monitoring the activities of therapeutic agents.

An important application of the microarray method allows for the assessment of differential gene expression in pairs of mRNA samples from two different tissues, or in the same tissue comparing normal versus disease states or time progression of the disease. Microarray analysis allows one to analyze the expression of known genes of interest, or to discover novel genes expressed differentially in tissues of interest. Thus, an attractive application of this technology is as a fundamental discovery tool to identify new genes, and their corresponding expression products, which contribute to the pathogenesis of disease and related conditions.

Microarray technology has been successfully applied to large-scale analysis of human gene expression to identify cancer-specific genes and inflammatory-specific genes (DeRisi et al., Nat. Genet., 14(4):457-60 [1996]; Heller et al., Proc. Natl. Acad. Sci. USA, 94(6):2150-55 [1997]). DeRisi et al. examined a pre-selected set of 870 different genes for their expression in a melanoma cell line and a non-tumorigenic version of the same cell line. The microarray analysis revealed a decrease in expression for 15/870 (1.7%) and an increase in expression for 63/870 (7.3%) of the genes in non-tumorigenic relative to tumorigenic cells (differential expression values <0.52 or >2.4 were deemed significant). Heller et al. employed microarrays to evaluate the expression of 1000 genes in cells taken from normal and inflamed human tissues. The results indicated that altered expression was evident in genes encoding inflammatory mediators such as IL-3, and a tissue metalloprotease. These results illustrate the utility of applying microarray technology to complex human diseases.

It would be beneficial to discover differentially expressed genes that are related to diseases or various disease states. It would further be beneficial to develop methods and compositions for the diagnostic evaluation and prognosis of conditions involving such diseases, for the identification of subjects exhibiting a predisposition to such conditions, for modulating the effect of these differentially expressed genes and their expression products, for monitoring patients undergoing clinical evaluation for the prevention and treatment of a disease, specifically cardiac, kidney or inflammatory disease, and for monitoring the efficacy of compounds used in clinical trials.

Secreted proteins mediate key biological processes including cell to cell interactions as well as important cellular functions such as cell growth and differentiation, and most protein-based drugs are secreted proteins including insulin, growth hormone, interferons, tissue plasminogen activator (tPA), and erythropoietin (EPO). It would, therefore, be particularly desirable to identify novel differentially expressed genes encoding secreted proteins.

### SUMMARY OF THE INVENTION

In one aspect, the present invention concerns an isolated nucleic acid molecule comprising a poly- or oligonucleotide selected from the group consisting of:
(a) a polynucleotide encoding a polypeptide having at least about 80% sequence identity with amino acids amino acids 22 to 122 of SEQ ID NO:1;
(b) a polynucleotide encoding a polypeptide having at least about 80% sequence identity with amino acids 56 to 122 of SEQ ID NO:1;
(c) a polynucleotide encoding amino acids 22 to 275 of SEQ ID NO: 1, or a transmembrane domain (membrane spanning segment/region) deleted or inactivated variant thereof;
(d) a polynucleotide hybridizing under stringent conditions with the complement of the coding region of SEQ ID NO: 2, and encoding at least one biological activity of the polypeptide encoded by clone P00210_D09 (SEQ ID NO: 2);
(e) a polynucleotide encoding at least about 50 contiguous amino acids from amino acids 22 to 122 of SEQ ID NO:1, wherein said polynucleotide encodes a polypeptide having at least one biological activity of the polypeptide encoded by clone P00210_D09 (SEQ ID NO: 2);
(f) a polynucleotide encoding at least about 50 contiguous amino acids from amino acids 56 to 122 of SEQ ID NO:1, wherein said polynucleotide encodes a polypeptide having at least one biological activity of the polypeptide encoded by clone P00210_D09 (SEQ ID NO: 2);
(g) a polynucleotide of SEQ ID NO:2;
(h) the complement of a polynucleotide of (a)-(g); and
(i) an antisense oligonucleotide capable of hybridizing with, and inhibiting the translation of, the mRNA encoded by a gene encoding a polypeptide of SEQ ID NO:1, or another mammalian (e.g. human) homologue thereof.

In another aspect, the invention concerns a vector comprising any of the poly- or oligonucleotides of (a)-(i) above.

In a further aspect, the invention concerns a recombinant host cell transformed with nucleic acid comprising any of the poly- or oligonucleotides of (a)-(i) above, or with a vector comprising any of the poly- or oligonucleotides of (a)-(i) above.

In a still further aspect, the invention concerns a recombinant method for producing a polypeptide by culturing a recombinant host cell transformed with nucleic acid comprising any of the polynucleotides of (a)-(g) above under conditions such that the polypeptide is expressed, and isolating the polypeptide.

In a different aspect, the invention concerns a polypeptide comprising:
(a) a polypeptide having at least about 80% identity with amino acids 22 to 122 of SEQ ID NO:1; or
(b) a polypeptide encoded by nucleic acid hybridizing under stringent conditions with the complement of the coding region of SEQ ID NO:2;
the polypeptides of (a) and (b) having at least one biological activity of the polypeptide encoded by clones P00210_D09 (SEQ ID NO:2).

In another aspect, the invention concerns a composition comprising a polypeptide as hereinabove defined in admixture with a pharmaceutically acceptable carrier. In a specific embodiment, the composition is a pharmaceutical composition, preferably for the treatment of a cardiac, renal or inflammatory disease, comprising an effective amount of a polypeptide of the present invention.

In yet another aspect, the invention concerns an antibody specifically binding a polypeptide of the present invention (as hereinabove defined).

In a further aspect, the invention concerns an antagonist or agonist of a polypetide of the present invention.

In a still further aspect, the invention concerns a composition, preferably a pharmaceutical composition, comprising an effective amount of an antibody herein, in admixture with a pharmaceutically acceptable carrier.

The invention further concerns a composition, preferably a pharmaceutical composition, comprising an effective amount of an antagonist or agonist of the present invention, in admixture with a pharmaceutically acceptable carrier.

In a further aspect, the invention concerns a method for the treatment of a cardiac, renal or inflammatory disease, comprising administering to a patient in need an effective amount of a polypeptide of the present invention or an antagonist or agonist thereof.

In a different aspect, the invention concerns a method for the treatment of a cardiac, renal or inflammatory disease, comprising administering to a patient in need an effective amount of a poly- or oligonucleotide of the present invention (as hereinabove defined).

The invention also concerns a method for the treatment of a cardiac, renal or inflammatory disease, comprising administering to a patient in need an effective amount of an antibody specifically binding to a polypeptide of the present invention.

In a further aspect, the invention concerns a method for screening a subject for a cardiac, renal or inflammatory disease characterized by the differential expression of the endogenous homologue of the protein of SEQ ID NO:1, comprising the steps of:
measuring the expression in the subject of the endogenous homologue of the protein of SEQ ID NO:1; and
determining the relative expression of such endogenous homologue in the subject compared to its expression in normal subjects, or compared to its expression in the same subject at an earlier stage of development of the cardiac, renal or inflammatory disease. The subject is preferably human and, accordingly, the endogenous protein is a human homologue of the rat protein of SEQ ID NO:1.

In a still further aspect, the invention concerns an array comprising one or more oligonucleotides complementary to reference RNA or DNA encoding a protein of SEQ ID NO:1 or another mammalian (e.g. human) homologue thereof, where the reference DNA or RNA sequences are obtained from both a biological sample from a normal subject and a biological sample from a subject exhibiting a cardiac, renal, or inflammatory disease, or from biological samples taken at different stages of a cardiac, renal, or inflammatory disease.

In yet another aspect, the invention concerns a method for detecting cardiac, kidney, or inflammatory disease in a human patient comprising the steps of:
providing an array of oligonucleotides at known locations on a substrate, which array comprises oligonucleotides complementary to reference DNA or RNA sequences encoding a human homologue of the protein of SEQ ID NO:1, where the reference DNA or RNA sequences are obtained from both a biological sample from a normal patient and a biological sample from a patient potentially exhibiting cardiac, renal, or inflammatory disease, or from a patient exhibiting cardiac, renal, or inflammatory disease, taken at different stages of such disease jointly referred to as “the test patient”);
exposing the array, under hybridization conditions, to a first sample of cDNA probes constructed from mRNA obtained from a biological sample from a corresponding biological sample of a normal patient or from a test patient at a certain stage of the disease;
exposing the array, under hybridization conditions, to a second sample of cDNA probes constructed from mRNA obtained from a biological sample obtained from the test patient (if the first sample was taken at a certain stage of the disease, the second sample is taken at a different stage of the disease);
quantifying any hybridization between the first sample of cDNA probes and the second sample of cDNA probes with the oligonucleotide probes on the array; and
determining the relative expression of genes encoding the human homologue of the protein of SEQ ID NO:1 in the biological samples from the normal patient and the test patient, or in the biological samples taken from the test patient at different stages of the disease.

The invention further concerns a diagnostic kit comprising an array herein (as defined above) for detecting and diagnosing a disease, specifically cardiac, kidney or inflammatory disease. This kit may comprise control oligonucleotide probes, PCR reagents and detectable labels. In addition, this kit may comprise biological samples taken from human subjects, said samples comprising blood or tissue, preferably cardiac tissue, more preferably left ventricle cells. Such diagnostic kits may also comprise antibodies (including poly- and monoclonal antibodies) to a polypeptide of the present invention, including the polypeptide of SEQ ID NO:1 and further mammalian (e.g. human) homologues thereof.

### BRIEF DESCRIPTION OF THE FIGURES

FIG. 1 (SEQ ID NO:1) shows the deduced amino acid sequence of the polypeptide encoded by the clone P00210_D09. The open reading frame (ORF) of the polypeptide contains 275 amino acid residues, of which the first 21 residues, including the initiating methionine, show the characteristis of a putative signal sequence, which is underlined. The sequence includes two putative membrane spanning segments at positions 35-55 and 123-143, respectively, which are boxed in the sequence.

FIG. 2 shows the nucleotide sequence of the clone P00210_D19 (SEQ ID NO: 3), in alignment with the encoded amino acid sequence, where the inititating methionine is circled. The total length of this sequence is 1031 bases, and the sequence encoded by the open reading frame (275 amino acid polypeptide, SEQ ID NO:1) is bracketed in the Figure. The complementary strand is also depicted (SEQ ID NO:19).

FIG. 3 shows the results of Northern blot analysis of P00210_D19 expression in rat heart, brain, spleen, lung, liver, skeletal muscle, and kidney tissue. P00210_DI 9 encodes a rare message, a putative about 900 bp transcript is detected in rat heart using polyA+ mRNA.

FIG. 4 shows the results of quantitative real-time PCR analysis of P00210_D19 expression in treated rat cardiac myocytes. Myocytes were treated with cardiotropin-1 (CT-I), phenylephrine, endothelin 1 (Eth-1), angiotension II (Ang2), transforming growth factor beta (TGFβ), tumor necorosis factor alpha (TNFa) and interleukin-1β (IL-1β). Panel A shows P00210_D09 expression after 24 hours of treatment. Expression was normalized to 18S ribosomal RNA expression after 24 hours of treatment. Expression was normalized to 18S ribosomal RNA expression. Treatment with CT-1, Phe, Eth-1, Ang2, TGFβ and TNFα for 2 hours decreased expression of P00210_D09 mRNA 2 to 3-fold. Treatment with CT-1, Phe, and TNF a for 24 hours decreased expression of P00210_D09 mRNA 1.8 fold.

FIG. 5 shows the tissue distribution of P00210_D09 RNA expression in rat as determined by quantitiative real-time PCR. Distribution was analyzed in brain, heart, kidney, liver, lung, skeletal muscle, spleen and testis. Predominant expression of P00210_D09 mRNA was seen in the heart. Significant expression was also observed in the brain and skeletal muscle.

FIG. 6 shows the expression profile of P00210_D09 RNA in the rat myocardial infarction model as determined by quantitative real-time PCR. Panel A shows P00210_D09 RNA expression in the left ventricle (LV) two weeks (LV2), four weeks (LV4), eight weeks (LV8) and twenty-two weeks (LV22) after surgically induced myocardial infarctin or a sham operation (SHAM). Panel B shows P00210_D09 RNA expression in the septim (Spt) under the indentical conditions. Expression was normalized to 18S ribosomal RNA expression. Significant induction of P00210_D09 was observed at 2 weeks in the Spt, 4 weeks in the LV and Spt and 8 weeks in the LV.

### DETAILED DESCRIPTION OF THE INVENTION

Definitions

Unless defined otherwise, technical and scientific terms used herein have the same meaning as commonly understood by one of ordinary skill in the art to which this invention belongs. Singleton et al., Dictionary of Microbiology and Molecular Biology 2^{nd} ed., J. Wiley & Sons (New York, N.Y. 1994), and March, Advanced Organic Chemistry Reactions, Mechanisms and Structure 4^{th} ed., John Wiley & Sons (New York, N.Y. 1992), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “polynucleotide”, when used in singular or plural, generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes DNAs and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

The term “polypeptide”, in singular or plural, is used herein to refer to any peptide or protein comprising two or more amino acids joined to each other in a linear chain by peptide bonds. As used herein, the term refers to both short chains, which also commonly are referred to in the art as peptides, oligopeptides and oligomers, and to longer chains, commonly referred to in the art as proteins. Polypeptides, as defined herein, may contain amino acids other than the 20 naturally occurring amino acids, and may include modified amino acids. The modification can be anywhere within the polypeptide molecule, such as, for example, at the terminal amino acids, and may be due to natural processes, such as processing and other post-translational modifications, or may result from chemical and/or enzymatic modification techniques which are well known to the art. The known modifications include, without limitation, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Such modifications are well known to those of skill and have been described in great detail in the scientific literature, such as, for instance, Creighton, T. E., Proteins—Structure And Molecular Properties, 2^{nd} Ed., W. H. Freeman and Company, New York (1993); Wold, F., “Posttranslational Protein Modifications: Perspectives and Prospects,” in Posttranslational Covalent Modification of Proteins, Johnson, B. C., ed., Academic Press, New York (1983), pp. 1-12; Seifter et al., “Analysis for protein modifications and nonprotein cofactors,” Meth. Enzymol. 182:626-646 (1990), and Rattan et al., Ann. N.Y. Acad. Sci. 663:48-62 (1992).

Modifications can occur anywhere in a polypeptide, including the peptide backbone, the amino acid side-chains and the amino or carboxyl termini. In fact, blockage of the amino or carboxyl group in a polypeptide, or both, by a covalent modification, is common in naturally occurring and synthetic polypeptides and such modifications may be present in polypeptides of the present invention, as well. For instance, the amino terminal residue of polypeptides made in E. coli, prior to proteolytic processing, almost invariably will be N-formylmethionine.

Modifications that occur in a polypeptide often will be a function of how the polypeptide is made. For polypeptides made by expressing a cloned gene in a host, for instance, the nature and extent of the modifications in large part will be determined by the host cell posttranslational modification capacity and the modification signals present in the polypeptide amino acid sequence. For instance, it is well known that glycosylation usually does not occur in certain bacterial hosts such as E. coli. Accordingly, when glycosylation is desired, a polypeptide is expressed in a glycosylating host, generally eukaryotic host cells. Insect cells often carry out the same posttranslational glycosylations as mammalian cells and, for this reason, insect cell expression systems have been developed to express efficiently mammalian proteins having native patterns of glycosylation.

It will be appreciated that the same type of modification may be present in the same or varying degree at several sites in a given polypeptide. Also, a given polypeptide may contain many types of modifications.

It will be appreciated that polypeptides are not always entirely linear. For instance, polypeptides may be branched as a result of ubiquitination, and they may be circular, with or without branching, generally as a result of posttranslational events, including natural processing and events brought about by human manipulation which do not occur naturally. Circular, branched and branched circular polypeptides may be synthesized by non-translation natural process and by entirely synthetic methods, as well. Such structures are within the scope of the polypeptides as defined herein.

The term “amino acid sequence variant” refers to molecules with some differences in their amino acid sequences as compared to a reference (e.g. native sequence) polypeptide. The amino acid alterations may be substitutions, insertions, deletions or any desired combinations of such changes in a native amino acid sequence.

Substitutional variants are those that have at least one amino acid residue in a native sequence removed and a different amino acid inserted in its place at the same position. The substitutions may be single, where only one amino acid in the molecule has been substituted, or they may be multiple, where two or more amino acids have been substituted in the same molecule.

Insertional variants are those with one or more amino acids inserted immediately adjacent to an amino acid at a particular position in a native amino acid sequence. Immediately adjacent to an amino acid means connected to either the α-carboxy or α-amino functional group of the amino acid.

Deletional variants are those with one or more amino acids in the native amino acid sequence removed. Ordinarily, deletional variants will have one or two amino acids deleted in a particular region of the molecule.

The amino acid sequence variants within the scope of the present invention may contain amino acid alterations, including substitutions and/or insertions and/or deletions in any region of the polypeptide of SEQ ID NO:1, including the N- and C-terminal regions. The amino acid sequence variants of the present invention show at least about 75%, more preferably at least about 85%, even more preferably at least about 90%, most preferably at least about 95% amino acid sequence identity with a polypeptide of SEQ ID NO:1 or with a native homologue thereof in another mammalian species, including humans.

“Sequence identity” is defined as the percentage of amino acid residues in a candidate sequence that are identical with the amino acid residues in a native polypeptide sequence, after aligning the sequences and introducing gaps, if necessary, to achieve the maximum percent sequence identity, and not considering any conservative substitutions as part of the sequence identity. The % sequence identity values are generated by the NCBI BLAST2.0 software as defined by Altschul et al., (1997), “Gapped BLAST and PSI-BLAST: a new generation of protein database search programs”, Nucleic Acids Res., 25:3389-3402. The parameters are set to default values, with the exception of the Penalty for mismatch, which is set to −1.

“Stringent” hybridization conditions are sequence dependent and will be different with different environmental parameters (e.g., salt concentrations, and presence of organics). Generally, stringent conditions are selected to be about 5° C. to 20° C. lower than the thermal melting point (Tₘ) for the specific nucleic acid sequence at a defined ionic strength and pH. Preferably, stringent conditions are about 5° C. to 10° C. lower than the thermal melting point for a specific nucleic acid bound to a complementary nucleic acid. The Tₘ is the temperature (under defined ionic strength and pH) at which 50% of a nucleic acid (e.g., tag nucleic acid) hybridizes to a perfectly matched probe

“Stringent” wash conditions are ordinarily determined empirically for hybridization of each set of tags to a corresponding probe array. The arrays are first hybridized (typically under stringent hybridization conditions) and then washed with buffers containing successively lower concentrations of salts, or higher concentrations of detergents, or at increasing temperatures until the signal to noise ratio for specific to non-specific hybridization is high enough to facilitate detection of specific hybridization. Stringent temperature conditions will usually include temperatures in excess of about 30° C., more usually in excess of about 37° C., and occasionally in excess of about 45° C. Stringent salt conditions will ordinarily be less than about 1000 mM, usually less than about 500 mM, more usually less than about 400 mM, typically less than about 300 mM, preferably less than about 200 mM, and more preferably less than about 150 mM. However, the combination of parameters is more important than the measure of any single parameter. See, e.g., Wetmur et al., J. Mol. Biol. 31:349-70 (1966), and Wetmur, Critical Reviews in Biochemistry and Molecular Biology 26(34):227-59 (1991). In a preferred embodiment, “stringent conditions” or “high stringency conditions,” as defined herein, may be hybridization in 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

As used herein, the term “polynucleotide encoding a polypeptide” and grammatical equivalents thereof, encompass polynucleotides which include a sequence encoding a polypeptide of the present invention, including polynucleotides that comprise a single continuous region or discontinuous regions encoding the polypeptide (for example, interrupted by introns) together with additional regions, that also may contain coding and/or non-coding sequences.

“Antisense oligodeoxynucleotides” or “antisense oligonucleotides” (which terms are used interchangeably) are defined as nucleic acid molecules that can inhibit the transcription and/or translation of target genes in a sequence-specific manner. The term “antisense” refers to the fact that the nucleic acid is complementary to the coding (“sense”) genetic sequence of the target gene. Antisense oligonucleotides hybridize in an antiparallel orientation to nascent mRNA through Watson-Crick base-pairing. By binding the target mRNA template, antisense oligonucleotides block the successful translation of the encoded protein. The term specifically includes antisense agents called “ribozymes” that have been designed to induce catalytic cleavage of a target RNA by addition of a sequence that has natural self-splicing activity (Warzocha and Wotowiec, “Antisense strategy: biological utility and prospects in the treatment of hematological malignancies.” Leuk. Lymphoma 24:267-281 [1997]).

The terms “vector”, “polynucleotide vector”, “construct” and “polynucleotide construct” are used interchangeably herein. A polynucleotide vector of this invention may be in any of several forms, including, but not limited to, RNA, DNA, RNA encapsulated in a retroviral coat, DNA encapsulated in an adenovirus coat, DNA packaged in another viral or viral-like form (such as herpes simplex, and adeno-associated virus (AAV)), DNA encapsulated in liposomes, DNA complexed with polylysine, complexed with synthetic polycationic molecules, conjugated with transferrin, complexed with compounds such as polyethylene glycol (PEG) to immunologically “mask” the molecule and/or increase half-life, or conjugated to a non-viral protein. Preferably, the polynucleotide is DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

The term “antagonist” is used in the broadest sense and includes any molecule that partially or fully blocks, inhibits or neutralizes a biological activity exhibited by a polypeptide of the present invention. In a similar manner, the term “agonist” is used in the broadest sense and includes any molecule that mimics a biological activity exhibited by a polypeptide of the present invention, for example, by specifically changing the function or expression of such polypeptide, or the efficiency of signaling through such polypeptide, thereby altering (increasing or inhibiting) an already existing biological activity or triggering a new biological activity.

The term “recombinant” when used with reference to a cell, animal, or virus indicates that the cell, animal, or virus encodes a foreign DNA or RNA. For example, recombinant cells optionally express nucleic acids (e.g., RNA) not found within the native (non-recombinant) form of the cell.

The term “antibody” is used in the broadest sense and specifically covers monoclonal antibodies (including agonist, antagonist, and neutralizing antibodies), polyclonal antibodies, multi-specific antibodies (e.g., bispecific antibodies), as well as antibody fragments. The monoclonal antibodies specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 [1984]). The monoclonal antibodies further include “humanized” antibodies or fragments thereof (such as Fv, Fab, Fab′, F(ab′)₂ or other antigen-binding subsequences of antibodies) which contain minimal sequence derived from non-human immunoglobulin. For the most part, humanized antibodies are human immunoglobulins (recipient antibody) in which residues from a CDR of the recipient are replaced by residues from a CDR of a non-human species (donor antibody) such as mouse, rat or rabbit having the desired specificity, affinity, and capacity. In some instances, Fv FR residues of the human immunoglobulin are replaced by corresponding non-human residues. Furthermore, humanized antibodies may comprise residues which are found neither in the recipient antibody nor in the imported CDR or framework sequences. These modifications are made to further refine and maximize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature 321:522-525 (1986); and Reichmann et al., Nature 332:323-329 (1988). The humanized antibody includes a PRIMATIZED® antibody wherein the antigen-binding region of the antibody is derived from an antibody produced by immunizing macaque monkeys with the antigen of interest.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (Zapata et al., Protein Eng. 8(10):1057-1062 (1995)); single-chain antibody molecules; and multispecific antibodies formed from antibody fragments.

The terms “differentially expressed gene,” “differential gene expression” and their synonyms, which are used interchangeably, refer to a gene whose expression is activated to a higher or lower level in a subject suffering from a disease, specifically a cardiac, kidney or inflammatory disease state, relative to its expression in a normal or control subject. The terms also include genes whose expression is activated to a higher or lower level at different stages of the same disease. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example. Differential gene expression may include a comparison of expression between two or more genes, or a comparison of the ratios of the expression between two or more genes, or even a comparison of two differently processed products of the same gene, which differ between normal subjects and subjects suffering from a disease, specifically a cardiac, kidney or inflammatory disease state, or between various stages of the same disease. Differential expression includes both quantitative, as well as qualitative, differences in the temporal or cellular expression pattern in a gene or its expression products among, for example, normal and diseased cells, or among cells which have undergone different disease events or disease stages. For the purpose of this invention, “differential gene expression” is considered to be present when there is at least an about 1.4-fold, preferably at least about 1.8-fold, more preferably at least about 2.0-fold, most preferably at least about 2.5-fold difference between the expression of a given gene in normal and diseased subjects, or in various stages of disease development in a diseased subject.

“Cardiac disease” includes congestive heart failure, myocarditis, dilated congestive cardiomyopathy, hypertrophic cardiomyopathy, restrictive cardiomyopathy, mitral valve disease, aortic valve disease, tricuspid valve disease, angina pectoris, myocardial infarction, cardiac arrhythmia, pulmonary hypertension, arterial hypertension, renovascular hypertension, arteriosclerosis, atherosclerosis, and cardiac tumors, along with any disease or disorder that relates to the cardiovascular system and related disorders, as well as symptoms indicative of, or related to, cardiac disease and related disorders.

As used herein, “heart failure” refers to an abnormality of cardiac function where the heart does not pump blood at the rate needed for the requirements of metabolizing tissues. The heart failure can be caused by any number of factors, including ischemic, congenital, rheumatic, or idiopathic forms.

As used herein “congestive heart failure” refers to a syndrome characterized by left ventricular dysfunction, reduced exercise tolerance, impaired quality of life, and markedly shortened life expectancy. Decreased contractility of the left ventricle leads to reduced cardiac output with consequent systemic arterial and venous vasoconstriction. This vasoconstriction, which appears to be mediated, in part, by the renin-angiotensis system, promotes the vicious cycle of further reductions of stroke volume followed by an increased elevation of vascular resistance.

As used herein “infarct” refers to an area of necrosis resulting from an insufficiency of blood supply. “Myocardial infarction” refers to myocardial necrosis resulting from the insufficiency of coronary blood supply.

“Kidney disease” includes acute renal failure, glomerulonephritis, chronic renal failure, azotemia, uremia, immune renal disease, acute nephritic syndrome, rapidly progressive nephritic syndrome, nephrotic syndrome, Berger's Disease, chronic nephritic/proteinuric syndrome, tubulointerstital disease, nephrotoxic disorders, renal infarction, atheroembolic renal disease, renal cortical necrosis, malignant nephroangiosclerosis, renal vein thrombosis, renal tubular acidosis, renal glucosuria, nephrogenic diabetes insipidus, Bartter's Syndrome, Liddle's Syndrome, polycystic kidney disease, medullary cystic disease, medullary sponge kidney, hereditary nephritis, and nail-patella syndrome, along with any disease or disorder that relates to the renal system and related disorders, as well as symptoms indicative of, or related to, renal or kidney disease and related disorders.

The phrases “polycystic kidney disease” “PKD” and “polycystic renal disease” are used interchangeably, and refer to a group of disorders characterized by a large number of cysts distributed throughout dramatically enlarged kidneys. The resultant cyst development leads to impairment of kidney function and can eventually cause kidney failure. “PKD” specifically includes autosomal dominant polycystic kidney disease (ADPKD) and recessive autosomal recessive polycystic kidney disease (ARPKD), in all stages of development, regardless of the underlying cause.

“Inflammatory disease” includes myocarditis, asthma, chronic inflammation, autoimmune diabetes, tumor angiogenesis, rheumatoid arthritis (RA), rheumatoid spondylitis, osteoarthritis, gouty arthritis and other arthritic conditions, sepsis, septic shock, endotoxic shock, Gram-negative sepsis, toxic shock syndrome, asthma, adult respiratory distress syndrome, stroke, reperfusion injury, CNS injuries such as neural trauma and ischemia, psoriasis restenosis, cerebral malaria, chronic pulmonary inflammatory disease, silicosis, pulmonary sarcosis, bone resorption diseases such as osteoporosis, graft versus host reaction, Crohn's Disease, ulcerative colitis including inflammatory bowel disease (IBD), Alzheimer's disease, and pyresis, along with any disease or disorder that relates to inflammation and related disorders, as well as symptoms indicative of, or related to, inflammation and related disorders.

The terms “treat” or “treatment” refer to both therapeutic treatment and prophylactic or preventative measures, wherein the object is to prevent or slow down (lessen) an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

“Chronic” administration refers to administration of the agent(s) in a continuous mode as opposed to an acute mode, so as to maintain the desired effect for an extended period of time.

“Intermittent” administration is treatment that is not consecutively done without interruption, but rather is cyclic in nature.

Administration “in combination with” one or more further therapeutic agents includes simultaneous (concurrent) and consecutive administration in any order.

An “individual” is a vertebrate, preferably a mammal, more preferably a human.

“Mammal” for purposes of treatment refers to any animal classified as a mammal, including humans, domestic and farm animals, and zoo, sports, or pet animals, such as dogs, horses, cats, cows, etc. Preferably, the mammal herein is human.

An “effective amount” is an amount sufficient to effect beneficial or desired therapeutic (including preventative) results. An effective amount can be administered in one or more administrations.

“Active” or “activity” means a qualitative biological and/or immunological property.

The phrase “immunological property” means immunological cross-reactivity with at least one epitope of the reference (native sequence) polypeptide molecule, wherein, “immunological cross-reactivity” means that the candidate polypeptide is capable of competitively inhibiting the qualitative biological activity of the reference (native sequence) polypeptide. The immunological cross-reactivity is preferably “specific”, which means that the binding affinity of the immunologically cross-reactive molecule identified to the corresponding polypeptide is significantly higher (preferably at least about 2-times, more preferably at least about 4-times, most preferably at least about 6-times higher) than the binding affinity of that molecule to any other known native polypeptide.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

Modes of Carrying Out the Invention

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, “Molecular Cloning: A Laboratory Manual”, 2nd edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4th edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in rImmunology” (J. E. Coligan et al., eds., 1991).

1. Identification of Differential Gene Expression and Further Characterization of Differentially Expressed Genes

The present invention is based on the identification of genes that are differentially expressed in the left ventricle in the Myocardial Infarction Model, as described in the Examples. Such models of differential gene expression can be utilized, among other things, for the identification of genes which are differentially expressed in normal cells versus cells in a disease state, specifically cardiac, kidney or inflammatory disease state, in cells within different diseases, among cells within a single given disease state, in cells within different stages of a disease, or in cells within different time stages of a disease.

Once a particular differentially expressed gene has been identified through the use of one model, its expression pattern can be further characterized, for example, by studying its expression in a different model. A gene may be regulated one way, i.e., the gene can exhibit one differential gene expression pattern, in a given model, but can be regulated differently in another model. The use, therefore, of multiple models can be helpful in distinguishing the roles and relative importance of particular genes in a disease, specifically cardiac, kidney or inflammatory disease.

a. In Vitro Models of Differential Gene Expression

A suitable model that can be utilized within the context of the present invention to discover differentially expressed genes is the in vitro specimen model. In a preferred embodiment, the specimen model uses biological samples from subjects, e.g., peripheral blood, cells and tissues, including surgical and biopsy specimens. Such specimens can represent normal peripheral blood and tissue or peripheral blood and tissue from patients suffering from a disease, specifically cardiac, kidney or inflammatory disease, or having undergone surgical treatment for disorders involving a disease, such as, for example, coronary bypass surgery. Surgical specimens can be procured under standard conditions involving freezing and storing in liquid nitrogen (see Karmali et al., Br. J. Cancer 48:689-96 [1983]). RNA from specimen cells is isolated by, for example, differential centrifugation of homogenized tissue, and analyzed for differential expression relative to other specimen cells, preferably using microarray analysis.

Cell lines can also be used to identify genes that are differentially expressed in a disease, specifically cardiac, kidney or inflammatory disease. Differentially expressed genes are detected, as described herein, by comparing the pattern of gene expression between the experimental and control conditions. In such models, genetically matched disease cell lines (e.g., variants of the same cell line) may be utilized. For example, the gene expression pattern of two variant cell lines can be compared, wherein one variant exhibits characteristics of one disease state while the other variant exhibits characteristics of another disease state.

Alternatively, two variant cell lines, both of which exhibit characteristics of the same disease, specifically cardiac, kidney or inflammatory disease, but which exhibit differing degrees of disease disorder severity may be used. Further, genetically matched cell lines can be utilized, one of which exhibits characteristics of a disease, specifically cardiac, kidney or inflammatory disease, state, while the other exhibits a normal cellular phenotype. In accordance with this aspect of the invention, the cell line variants are cultured under appropriate conditions, harvested, and RNA is isolated and analyzed for differentially expressed genes, as with the other models. In a preferred embodiment, microarray analysis is used.

b. In Vivo Models of Differential Gene Expression

In the in vivo model, animal models of a disease, specifically cardiac, kidney or inflammatory disease, and related disorders, can be utilized to discover differentially expressed gene sequences. The in vivo nature of such disease models can prove to be especially predictive of the analogous responses in living patients, particularly human patients. Animal models for a disease, specifically cardiac, kidney or inflammatory disease, which can be utilized for in vivo models include any of the animal models described below. In a preferred embodiment, RNA from both the normal and disease state model is isolated and analyzed for differentially expressed genes using microarray analysis.

As presented in the examples, three representative in vivo cardiac disease models, a representative kidney disease model, and a representative inflammatory disease model have been successfully utilized to identify differentially expressed genes, and are believed to be useful to further characterize the genes and polypeptides of the present invention. These genes are expressed at higher or lower levels in the disease state, relative to the normal state, and preferably are expressed at least about a two-fold higher or lower level relative to the normal state at at least one time point.

Representative in vivo animal models for use in the present invention include the following: general inflammation—carrageenan-induced paw edema, arachidonic acid-induced ear inflammation; arthritis—adjuvant-induced polyarthritis, collagen-induced arthritis, streptococcal cell wall-induced arthritis; multiple sclerosis—experimental autoimmune encephalomyelitis (EAE); Systemic Lupus Erythematosis (SLE); NZB—spontaneous SLE mouse, DNA/anti-DNA immune complex-induced SLE; insulin-dependent diabetes mellitus —NOD spontaneous diabetes mouse; inflammatory bowel disease—acetic acid or trinitrobenzene sulfonic (TNBS)-induced ulcerative colitis; respiratory disease—antigen-induced bronchoconstriction (asthma), lipopolysaccharide (LPS)-induced acute respiratory distress syndrome (ARDS); analgesia—acetic acid-induced or phenylquinone-induced writhing, latency of tail-withdrawal (hot plate); transplant organ rejection—allograft rejection (kidney, lung, heart)-acute and chronic arteriolsclerosis; kidney disease—unilateral nephrectomy (acute renal failure), cyclosporin-induced nephropathy, accelerated crescentic anti-glomerular basement membrane (GBM) glomerulonephritis, soluble immune complex-induced nephritis (see generally Aziz, Bioassays 17:8 703-12 [1995]); and cardiac disease—spontaneous cardiomyopathic hamsters (heart failure), myocardial infarction (MI) model. pacing-induced model of failure (Riegger model), arrhythmias following myocardial infarction (Harris model), aconitine/chloroform-induced arrhythmisa, carotid artery injury (restenosis), balloon angioplasty (restenosis). One skilled in the art understands that the present invention is not limited to the in vivo models recited above and that any known models can be used within the context of the present invention.

Microarray Technique

In a preferred embodiment of the present invention, microarrays are utilized to assess differential expression of genes. In one aspect of the present invention, DNA microarrays are utilized to assess the expression profile of genes expressed in normal subjects and subjects suffering from a disease, specifically cardiac, kidney or inflammatory disease. Identification of the differentially expressed disease genes can be performed by: constructing normalized and subtracted cDNA libraries from mRNA extracted from the cells or tissue of healthy animals and an animal model of disease or of healthy patients and diseased patients, for example, using any of the in vitro or in vivo models described above; purifying the DNA of cDNA libraries of clones representing healthy and diseased cells or tissue, microarraying the purified DNA for expression analysis; and probing microarrays to identify the genes from the clones that are differentially expressed using labeled cDNA from healthy and diseased cells or tissues.

In a specific embodiment of the microarray technique, PCR amplified inserts of cDNA clones are applied to a substrate in a dense array. Preferably at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(20):106-49 [1996]).

In a specific embodiment, in vivo models of disease states are used to detect differentially expressed genes. By way of example, three representative cardiac disease models, a representative kidney disease model, and a representative inflammatory disease model were successfully utilized to identify specific differentially expressed genes. Summarizing the representative general protocol used for such in vivo models, separate DNA libraries were constructed from mRNA extracted from disease state tissue and normal tissue. From these libraries, at least 20,000 unidentified cDNA clones were preferably chosen for analysis and microarrayed on chips. Probes generated from normal and disease tissue, from multiple time points, were hybridized to the microarray. By this approach, genes, which are differentially expressed in normal and diseased tissue, were revealed and further identified by DNA sequencing. The analysis of the clones for differential expression reveal genes whose expression is elevated or decreased in association with a disease, specifically cardiac, kidney or inflammatory disease, in the specific in vivo model chosen.

Further Characterization of Differentially Expressed Genes

The differentially expressed genes of the present invention, in particular the rat gene of SEQ ID NO: 2 and its further mammalian (e.g. human) equivalents, are screened to obtain more information about the biological function of such genes. This information can, in turn, lead to the designation of such genes or their gene products as potential therapeutic or diagnostic molecules, or targets for identifying such molecules.

The goal of the follow-up work after a differentially expressed gene has been identified is to identify its target cell type(s), function and potential role in disease pathology. To this end, the differentially expressed genes are screened to identify cell types responding to the gene product, to better understand the mechanism by which the identified cell types respond to the gene product, and to find known signaling pathways that are affected by the expression of the gene.

When further characterization of a differentially expressed gene indicates that a modulation of the gene's expression or a modulation of the gene product's activity can inhibit or treat a disease, specifically cardiac, kidney or inflammatory disease, the differentially expressed gene or its gene product becomes a potential drug candidate, or a target for developing a drug candidate for the treatment of a cardiac, kidney or inflammatory disease, or may be used as a diagnostic.

Where further characterization of a differentially expressed gene reveals that modulation of the gene expression or gene product cannot retard or treat a target disease, the differentially expressed gene may still contribute to developing a gene expression diagnostic pattern correlative of a disease or its disorders. Accordingly, such genes may be useful as diagnostics.

A variety of techniques can be utilized to further characterize the differentially expressed genes after they are identified.

First, the nucleotide sequence of the identified genes, which can be obtained by utilizing standard techniques well known to those of skill in the art, can be used to further characterize such genes. For example, the sequence of the identified genes can reveal homologies to one or more known sequence motifs, which can yield information regarding the biological function of the identified gene product.

Second, an analysis of the tissue or cell type distribution of the mRNA produced by the identified genes can be conducted, utilizing standard techniques well known to those of skill in the art. Such techniques can include, for example, Northern analyses, microarrays, real time (RT-coupled PCR), and RNase protection techniques. In a preferred embodiment, transcriptional screening is used, which may be based on the transfection of cells with an inducible promoter-luciferase plasmid construct, real time PCR, or microarrays, the real time PCR and microarray approached being particularly preferred. Such analyses provide information as to whether the identified genes are expressed in further tissues expected to contribute to a disease, specifically cardiac, kidney or inflammatory disease. These techniques can also provide quantitative information regarding steady state mRNA regulation, yielding data concerning which of the identified genes exhibits a high level of regulation preferably in tissues which can be expected to contribute to a disease state. Additionally, standard in situ hybridization techniques can be utilized to provide information regarding which cells within a given tissue express the identified gene. Specifically, these techniques can provide information regarding the biological function of an identified gene relative to a disease, specifically cardiac, kidney or inflammatory disease, where only a subset of the cells within the tissue is thought to be relevant to the disorder.

Third, the sequences of the identified differentially expressed genes can be used, utilizing standard techniques, to place the genes onto genetic maps, e.g., mouse (Copeland et al., Trends in Genetics 7:113-18 (1991)) and human genetic maps (Cohen et al., Nature 266:698-701 [1993]). This mapping information can yield information regarding the genes' importance to human disease by identifying genes that map within genetic regions to which known genetic disease disorders map.

After the follow-up screening is completed, relevant, targeted in vivo and in vitro systems can be used to more directly assess the biological function of the identified genes. In vivo systems can include animal systems that naturally exhibit symptoms of a disease, specifically cardiac, kidney or inflammatory disease, or ones engineered to exhibit such symptoms. Animals of any species, including, but not limited to, mice, rats, rabbits, guinea pigs, pigs, micro-pigs, goats, and non-human primates, e.g., baboons, monkeys, and chimpanzees, can be used to generate animal models of a disease, specifically cardiac, kidney or inflammatory disease. Any technique known in the art can be used to introduce a target gene transgene into animals to produce the founder lines of transgenic animals. Such techniques include, pronuclear microinjection (Hoppe et al., U.S. Pat. No. 4,873,191 (1989)); retrovirus mediated gene transfer into germ lines (Van der Fatten et al., Proc. Natl. Acad. Sci. USA 82:6148-52 (1985)); gene targeting in embryonic stem cells (Thompson et al., Cell 56:313-21 (1989)); electroporation of embryos (Lo, Mol. Cell. Biol. 3:1803-14 (1983)); and sperm-mediated gene transfer (Lavitrano et al., Cell 57:717-23 (1989)). For a review of such techniques, see Gordon, Intl. Rev. Cytol. 115:171-229 (1989). Further techniques will be detailed below, in connection with the gene therapy applications of the polynucleotides of the present invention.

The present invention provides for transgenic animals that carry the transgene in all their cells, as well as animals which carry the transgene in some, but not all their cells, i.e., mosaic animals. The transgene can be integrated, either as a single transgene or in concatamers, e.g., head-to-head tandems or head-to-tail tandems. The transgene can also be selectively introduced into and activated in a particular cell type by following, for example, the teaching of Lasko et al., Proc. Natl. Acad. Sci. USA 89:6232-36 (1992). The regulatory sequences required for such a cell-type specific activation depends upon the particular cell type of interest, and will be apparent to those of skill in the art.

When it is desired that the transgene be integrated into the chromosomal site of the endogenous target gene, gene targeting is preferred. Briefly, when such a technique is to be utilized, vectors containing some nucleotide sequences homologous to the endogenous target gene of interest are designed for the purpose of integrating, via homologous recombination with chromosomal sequences, into and disrupting the function of the nucleotide sequence of the endogenous target gene. The transgene can also be selectively introduced into a particular cell type, thus inactivating the endogenous gene of interest in only that cell type, by following the teaching of Gu et al. (Science 265:103-06 [1994]). The regulatory sequences required for such a cell-type specific inactivation depends upon the particular cell type of interest, and will be apparent to those of skill in the art.

Once transgenic animals have been generated, the expression of the recombinant target gene and protein can be assayed using standard techniques. Initial screening can be accomplished by Southern blot analysis or PCR techniques to analyze animal tissues to assay whether integration of the transgene has taken place. The level of mRNA expression of the transgene in the tissues of the transgenic animals can also be assessed using techniques which include Northern blot analysis of tissue samples obtained from the animal, in situ hybridization analysis, and RT-coupled PCR. Samples of target gene-expressing tissue can also be evaluated immunocytochemically using antibodies specific for the transgenic product of interest.

The transgenic animals that express target gene mRNA or target gene transgene peptide (detected immunocytochemically, using antibodies directed against target gene product epitopes) at easily detectable levels should then be further evaluated to identify those animals which display disease characteristics or symptoms. Additionally, specific cell types within the transgenic animals can be analyzed for cellular phenotypes characteristic of a disease, specifically cardiac, kidney or inflammatory disease. Such cellular phenotypes can include, for example, differential gene expression characteristic of cells within a given disease state of interest. Further, such cellular phenotypes can include an assessment of a particular cell type diagnostic pattern of expression and its comparison to known diagnostic expression profiles of the particular cell type in animals exhibiting a disease, specifically cardiac, kidney or inflammatory disease. Such transgenic animals serve as suitable models. Once transgenic founder animals are produced, they can be bred, inbred, outbred, or crossbred to produce colonies of the particular animal.

The animal models described above and in the Examples, can be used to generate cell lines for use in cell-based in vitro assays to further characterize the differentially expressed genes of the invention and their gene products. Techniques that can be used to derive a continuous cell line from transgenic animals are disclosed, for example, by Small et al., Mol. Cell Biol. 5:642-48 (1985).

Alternatively, cells of a cell type known to be involved in a cardiac, kidney or inflammatory disease can be transfected with sequences capable of increasing or decreasing the amount of target gene expression within the cell. For example, sequences of the differentially expressed genes herein can be introduced into, and overexpressed in, the genome of the cell of interest, or if endogenous target gene sequences are present, they can either be overexpressed or, be disrupted in order to underexpress or inactivate target gene expression.

The information obtained through such characterizations can suggest relevant methods for the treatment of a disease, specifically cardiac, kidney or inflammatory disease, involving the gene of interest. For example, treatment can include a modulation of gene expression or gene product activity. Characterization procedures such as those described herein can indicate where such modulation should involve an increase or a decrease in the expression or activity of the gene or gene product of interest.

2. Production of Polynucleotides and Polypeptides

The polypeptides of the present invention are preferably produced by techniques of recombinant

DNA technology. DNA encoding a native polypeptide herein, including the polypeptide of SEQ ID NO:1, can be obtained from cDNA libraries prepared from tissue believed to possess the corresponding mRNA and to express it at a detectable level. For example, cDNA library can be constructed by obtaining polyadenylated mRNA from a cell line known to express the desired polypeptide, and using the mRNA as a template to synthesize double-stranded cDNA. In the present case, a suitable source for the desired mRNA may be heart tissue obtained from normal heart or from the Myocardial Infarction Model (MI model) mentioned above, and described in detail in the Examples. The polypeptide genes of the present invention can also be obtained from a genomic library, such as a human genomic cosmid library.

Libraries, either cDNA or genomic, are screened with probes designed to identify the gene of interest or the protein encoded by it. For cDNA expression libraries, suitable probes include monoclonal and polyclonal antibodies that recognize and specifically bind to a polypeptide of SEQ ID NO:1 (encoded by the P00210_D09 gene of SEQ ID NO:2). For cDNA libraries, suitable probes include oligonucleotide probes (generally about 20-80 bases) that encode known or suspected portions of a polypeptide herein, from the same or different species, and/or complementary or homologous cDNAs or fragments thereof that encode the same or a similar gene. Appropriate probes for screening genomic libraries include, without limitation, oligonucleotides, cDNAs, or fragments thereof that encode the same or a similar gene, and/or homologous genomic DNAs or fragments thereof. Screening the cDNA and genomic libraries with the selected probe may be conducted using standard protocols as described, for example, in Chapters 10-12 of Sambrook et al., Molecular Cloning: A Laboratory Manual. New York, Cold Spring Harbor Laboratory Press (1989).

According to a preferred method, carefully selected oligonucleotide probes are used to screen cDNA libraries from various tissues, preferably from heart and/or kidney tissues. The oligonucleotide sequences selected as probes should be sufficient in length and sufficiently unique and unambiguous that false positives are minimized. The actual sequences can be designed based on regions of SEQ ID NO: 2 which have the least codon redundance. The oligonucleotides may be degenerate at one or more positions. The use of degenerate oligonucleotides is of particular importance where a library is screened from a species in which preferential codon usage is not known.

The oligonuleotides must be labeled such that they can be detected upon hybridization to DNA in the library screened. Preferably, the 5′ end of the oligonucleotide is radiolabeled, using APT (e.g. γ³²P) and polynucleotide kinase. However, other labeling, e.g. biotinylation or enzymatic labeling are also suitable.

Alternatively, to obtain DNA encoding a homologue of rat polypeptides specifically disclosed herein (SEQ ID NO:1) in another mammalian species, e.g. in humans, one only needs to conduct hybridization screening with labeled rat DNA (SEQ ID NO:2) or fragments thereof, selected following the principles outlined above, in order to detect clones which contain homologous sequences in the cDNA libraries obtained from appropriate tissues (e.g. heart or kidney) of the particular animal, such as human (cross-species hybridization). Full-length clones can then be identified, for example, by restriction endonuclease analysis and nucleic acid sequencing. If full-length clones are not identified, appropriate fragments are recovered from the various clones and ligated at restriction sites common to the fragments to assemble a full-length clone.
cDNAs encoding the polypeptides of the present invention can also be identified and isolated by other known techniques, such as by direct expression cloning or by using the PCR technique, both of which are well known are described in textbooks, such as those referenced hereinbefore.

Once the sequence is known, the nucleic acid encoding a particular polypeptide of the present invention can also be obtained by chemical synthesis, following known methods, such as the phosphoramidite method (Beaucage and Caruthers, Tetrahedron Letters 22:1859 [1981]; Matteucci and Caruthers, Tetrahedron Letters 21:719 [1980]; and Matteucci and Caruthers, J. Amer. Chem. Soc. 103: 3185 [1981]), and the phosphotriester approach (Ito et al., Nucleic Acids Res. 10:1755-1769 [1982]).

The cDNA encoding the desired polypeptide of the present invention is inserted into a replicable vector for cloning and expression. Suitable vectors are prepared using standard techniques of recombinant DNA technology, and are, for example, described in the textbooks cited above. Isolated plasmids and DNA fragments are cleaved, tailored, and ligated together in a specific order to generate the desired vectors. After ligation, the vector containing the gene to be expressed is transformed into a suitable host cell.

Host cells can be any eukaryotic or prokaryotic hosts known for expression of heterologous proteins.

The polypeptides of the present invention can be expressed in eukaryotic hosts, such as eukaryotic microbes (yeast), cells isolated from multicellular organisms (mammalian cell cultures), plants and insect cells.

While prokaryotic host provide a convenient means to synthesize eukaryotic proteins, when made this fashion, proteins usually lack many of the immunogenic properties, three-dimensional conformation, glycosylation, and other features exhibited by authentic eukaryotic proteins. Eukaryotic expression systems overcome these limitations.

Yeasts are particularly attractive as expression hosts for a number of reasons. They can be rapidly growth on inexpensive (minimal) media, the recombinant can be easily selected by complementation, expressed proteins can be specifically engineered for cytoplasmic localization or for extracellular export, and are well suited for large-scale fermentation.

Saccharomyces cerevisiae is the most commonly used among lower eukaryotic hosts. However, a number of other genera, species, and strains are also available and useful herein, such as Pichia pastoris (EP 183,070; Sreekrishna et al., J. Basic Microbiol. 28:165-278 [1988]). Yeast expression systems are commercially available, and can be purchased, for example, from Invitrogen (San Diego, Calif.). Other yeasts suitable for VEGF expression include, without limitation, Kluyveromyces hosts (U.S. Pat. No. 4,943,529), e.g. Kluyveromyces lactis; Schizosaccharomyces pombe (Beach and Nurse, Nature 290:140 (1981); Aspergillus hosts, e.g. A. niger (Kelly and Hynes, EMBO J. 4:475-479 [1985]) and A. nidulans (Ballance et al., Biochem. Biophys. Res. Commun. 112:284-289 [1983]), and Hansenula hosts, e.g. Hansenula polymorpha.

Preferably a methylotrophic yeast is used as a host in performing the methods of the present invention. Suitable methylotrophic yeasts include, but are not limited to, yeast capable of growth on methanol selected from the group consisting of the genera Pichia and Hansenula. A list of specific species which are exemplary of this class of yeasts may be found, for example, in C. Anthony, The Biochemistry of Methylotrophs, 269 (1982). Presently preferred are methylotrophic yeasts of the genus Pichia such as the auxotrophic Pichia pastoris GS115 (NRRL Y-15851); Pichia pastoris GS190 (NRRL Y-18014) disclosed in U.S. Pat. No. 4,818,700; and Pichia pastoris PPF1 (NRRL Y-18017) disclosed in U.S. Pat. No. 4,812,405. Auxotrophic Pichia pastoris strains are also advantageous to the practice of this invention for their ease of selection. It is recognized that wild type Pichia pastoris strains (such as NRRL Y-11430 and NRRL Y-11431) may be employed with equal success if a suitable transforming marker gene is selected, such as the use of SUC2 to transform Pichia pastoris to a strain capable of growth on sucrose, or if an antibiotic resistance marker is employed, such as resistance to G418. Pichia pastoris linear plasmids are disclosed, for example, in U.S. Pat. No. 5,665,600.

Suitable promoters used in yeast vectors include the promoters for 3-phosphoglycerate kinase (Hitzeman et al, J. Biol. Chem. 255:2073 [1980]); and other glycolytic enzymes (Hess et al, J. Adv. Enzyme Res. 7:149 [1968]; Holland et al., Biochemistry 17:4900 [1978]), e.g., enolase, glyceraldehyde-3-phosphate dehydrogenase, hexokinase, pyvurate decarboxylase, phosphofructokinase, glucose-6-phosphate isomerase, 3-phosphoglycerate mutase, pyruvate kinase, triosephosphate somerase, phosphoglucose isomerase, and glucokinase. In the constructions of suitable expression plasmids, the termination sequences associated with these genes are also ligated into the expression vector 3′ of the sequence desired to be expressed to provide polyadenylation of the mRNA and termination. Other promoters that have the additional advantage of transcription controlled by growth conditions are the promoter regions for alcohol oxidase 1 (AOX1, particularly preferred for expression in Pichia), alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Any plasmid vector containing a yeast-compatible promoter and termination sequences, with or without an origin of replication, is suitable. Yeast expression systems are commercially available, for example, from Clontech Laboratories, Inc. (Palo Alto, Calif., e.g. pYEX 4T family of vectors for S. cerevisiae), Invitrogen (Carlsbad, Calif., e.g. pPICZ series Easy Select Pichia Expression Kit) and Stratagene (La Jolla, Calif., e.g. ESP™ Yeast Protein Expression and Purification System for S. pombe and pESC vectors for S. cerevisiae).

Cell cultures derived from multicellular organisms may also be used as hosts to practice the present invention. While both invertebrate and vertebrate cell cultures are acceptable, vertebrate cell cultures, particularly mammalian cells, are preferable. Examples of suitable cell lines include monkey kidney CV1 cell line transformed by SV40 (COS-7, ATCC CRL 1651); human embryonic kidney cell line 293S (Graham et al, J. Gen. Virol. 36:59 [1977]); baby hamster kidney cells (BHK, ATCC CCL 10); Chinese hamster ovary (CHO) cells (Urlaub and Chasin, Proc. Natl. Acad. Sci. USA 77:4216 [1980]; monkey kidney cells (CVI-76, ATCC CCL 70); African green monkey cells (VERO-76, ATCC CRL-1587); human cervical carcinoma cells (HELA, ATCC CCL 2); canine kidney cells (MDCK, ATCC CCL 34); human lung cells (W138, ATCC CCL 75); and human liver cells (Hep G2, HB 8065).

Suitable promoters used in mammalian expression vectors are often of viral origin. These viral promoters are commonly derived from cytomeagolavirus (CMV), polyoma virus, Adenovirus2, and Simian Virus 40 (SV40). The SV40 virus contains two promoters that are termed the early and late promoters. They are both easily obtained from the virus as one DNA fragment that also contains the viral origin of replication (Fiers et al., Nature 273:113 [1978]). Smaller or larger SV40 DNA fragments may also be used, provided they contain the approximately 250-bp sequence extending from the HindIII site toward the Bgll site located in the viral origin of replication. An origin of replication may be obtained from an exogenous source, such as SV40 or other virus, and inserted into the cloning vector. Alternatively, the host cell chromosomal mechanism may provide the origin of replication. If the vector containing the foreign gene is integrated into the host cell chromosome, the latter is often sufficient.

Eukaryotic expression systems employing insect cell hosts may rely on either plasmid or baculoviral expression systems. The typical insect host cells are derived from the fall army worm (Spodoptera frugiperda). For expression of a foreign protein these cells are infected with a recombinant form of the baculovirus Autographa californica nuclear polyhedrosis virus which has the gene of interest expressed under the control of the viral polyhedrin promoter. Other insects infected by this virus include a cell line known commercially as “High 5” (Invitrogen) which is derived from the cabbage looper (Trichoplusia ni). Another baculovirus sometimes used is the Bombyx mori nuclear polyhedorsis virus which infect the silk worm (Bombyx mori). Numerous baculovirus expression systems are commercially available, for example, from Invitrogen (Bac-N-Blue™), Clontech (BacPAK™ Baculovirus Expression System), Life Technologies (BAC-TO-BAC™), Novagen (Bac Vector System™), Pharmingen and Quantum Biotechnologies). Another insect cell host is common fruit fly, Drosophila melanogaster, for which a transient or stable plasmid based transfection kit is offered commercially by Invitrogen (The DES™ System).

Prokaryotes are the preferred hosts for the initial cloning steps, and are particularly useful for rapid production of large amounts of DNA, for production of single-stranded DNA templates used for site-directed mutagenesis, for screening many mutants simultaneously, and for DNA sequencing of the mutants generated. E. coli strains suitable for the production of the polypeptides of the present invention include, for example, BL21 carrying an inducible T7 RNA polymerase gene (Studier et al., Methods Enzymol. 185:60-98 [1990]); AD494 (DE3); EB105; and CB (E. coli B) and their derivatives; K12 strain 214 (ATCC 31,446); W3110 (ATCC 27,325); X1776 (ATCC 31,537); HB101 (ATCC 33,694); JM101 (ATCC 33,876); NM522 (ATCC 47,000); NM538 (ATCC 35,638); NM539 (ATCC 35,639), etc. Many other species and genera of prokaryotes may be used as well. Prokaryotes, e.g. E. coli, produce the polypeptides of the present invention in an unglycosylated form.

Vectors used for transformation of prokaryotic host cells usually have a replication site, marker gene providing for phenotypic selection in transformed cells, one or more promoters compatible with the host cells, and a polylinker region containing several restriction sites for insertion of foreign DNA. Plasmids typically used for transformation of E. coli include pBR322, pUC18, pUCl9, pUC118, pUC119, and Bluescript M13, all of which are commercially available and described in Sections 1.12-1.20 of Sambrook et al., supra. The promoters commonly used in vectors for the transformation of prokaryotes are the T7 promoter (Studier et al., supra); the tryptophan (trp) promoter (Goeddel et al., Nature 281:544 [1979]); the alkaline phosphatase promoter (phoA); and the P-lactamase and lactose (lac) promoter systems. In E. coli, some polypeptides accumulate in the form of inclusion bodies, and need to be solubilized, purified, and refolded. These steps can be carried out by methods well known in the art.

Many eukaryotic proteins, including the polypeptide of SEQ ID NO:1 disclosed herein, contain an endogenous signal sequence as part of the primary translation product. This sequence targets the protein for export from the cell via the endoplasmic reticulum and Golgi apparatus. The signal sequence is typically located at the amino terminus of the protein, and ranges in length from about 13 to about 36 amino acids. Although the actual sequence varies among proteins, all known eukaryotic signal sequences contain at least one positively charged residue and a highly hydrophobic stretch of 10-15 amino acids (usually rich in the amino acids leucine, isoleucine, valine and phenylalanine) near the center of the signal sequence. The signal sequence is normally absent from the secreted form of the protein, as it is cleaved by a signal peptidase located on the endoplasmic reticulum during translocation of the protein into the endoplasmic reticulum. The protein with its signal sequence still attached is often referred to as the pre-protein, or the immature form of the protein, in contrast to the protein from which the signal sequence has been cleaved off, which is usually referred to as the mature protein. Proteins may also be targeted for secretion by linking a heterologous signal sequence to the protein. This is readily accomplished by ligating DNA encoding a signal sequence to the 5′ end of the DNA encoding the protein, and expressing the fusion protein in an appropriate host cell. Prokaryotic and eukaryotic (yeast and mammalian) signal sequences may be used, depending on the type of the host cell. The DNA encoding the signal sequence is usually excised from a gene encoding a protein with a signal sequence, and then ligated to the DNA encoding the protein to be secreted. Alternatively, the signal sequence can be chemically synthesized. The signal must be functional, i.e. recognized by the host cell signal peptidase such that the signal sequence is cleaved and the protein is secreted. A large variety of eukaryotic and prokaryotic signal sequences is known in the art, and can be used in performing the process of the present invention. Yeast signal sequences include, for example, acid phosphatase, alpha factor, alkaline phosphatase and invertase signal sequences. Prokaryotic signal sequences include, for example LamB, OmpA, OmpB and OmpF, MalE, PhoA, and β lactamase.

Mammalian cells are usually transformed with the appropriate expression vector using a version of the calcium phosphate method (Graham et al., Virology 52:546 [1978]; Sambrook et al., supra, sections 16.32-16.37), or, more recently, lipofection. However, other methods, e.g. protoplast fusion, electroporation, direct microinjection, etc. are also suitable.

Yeast hosts are generally transformed by the polyethylene glycol method (Hinnen, Proc. Natl. Acad. Sci. USA 75:1929 [1978]). Yeast, e.g. Pichiapastoris, can also be transformed by other methodologies, e.g. electroporation.

Prokaryotic host cells can, for example, be transformed using the calcium chloride method (Sambrook et al., supra, section 1.82), or electroporation.

More recently, techniques have been developed for the expression of heterologous proteins in the milk of non-human transgenic animals. For example, Krimpenfort et al., Biotechnology 9:844-847 (1991) describes microinjection of fertilized bovine oocytes with genes encoding human proteins and development of the resulting embryos in surrogate mothers. The human genes were fused to the bovine.alpha.S.sub. 1 casein regulatory elements. This general technology is also described in PCT Application WO91/08216 published Jun. 13, 1991. PCT application WO88/00239, published Jan. 14, 1988, describes procedures for obtaining suitable regulatory DNA sequences for the products of the mammary glands of sheep, including beta lactoglobulin, and the construction of transgenic sheep modified so as to secrete foreign proteins in milk. PCT publication WO88/01648, published Mar. 10, 1988, generally describes construction of transgenic animals which secrete foreign proteins into milk under control of the regulatory sequences of bovine alpha lactalbumin gene. PCT application WO88/10118, published Dec. 29, 1988, describes construction of transgenic mice and larger mammals for the production of various recombinant human proteins in milk. Thus, techniques for construction of appropriate host vectors containing regulatory sequences effective to produce foreign proteins in mammary glands and cause the secretion of said protein into milk are known in the art.

Among the milk-specific protein promoters are the casein promoters and the beta lactoglobulin promoter. The casein promoters may, for example, be selected from an alpha casein promoter, a beta casein promoter or a kappa casein promoter. Preferably, the casein promoter is of bovine origin and is an alpha S-1 casein promoter. Among the promoters that are specifically activated in mammary is the long terminal repeat (LTR) promoter of the mouse mammary tumor virus (MMTV). The milk-specific protein promoter or the promoters that are specifically activated in mammary tissue may be derived from either cDNA or genomic sequences. Preferably, they are genomic in origin.

Signal peptides that are useful in expressing heterologous proteins in the milk of transgenic mammals include milk-specific signal peptides or other signal peptides useful in the secretion and maturation of eukaryotic and prokaryotic proteins. Preferably, the signal peptide is selected from milk-specific signal peptides or the signal peptide of the desired recombinant protein product, if any. Most preferably, the milk-specific signal peptide is related to the milk-specific promoter used in the expression system of this invention.

The present invention includes amino acid sequence variants of the native rat polypeptide of SEQ ID NO:1 or its analogues in any other animal, e.g. mammalian species, including humans. Such amino acid sequence variants can be produced by expressing the underlying DNA sequence in a suitable recombinant host cell, as described above, or by in vitro synthesis of the desired polypeptide. The nucleic acid sequence encoding a polypeptide variant of the present invention is preferably prepared by site-directed mutagenesis of the nucleic acid sequence encoding the corresponding native (e.g. human) polypeptide. Particularly preferred is site-directed mutagenesis using polymerase chain reaction (PCR) amplification (see, for example, U.S. Pat. No. 4,683,195 issued 28 Jul. 1987; and Current Protocols In Molecular Biology, Chapter 15 (Ausubel et al., ed., 1991). Other site-directed mutagenesis techniques are also well known in the art and are described, for example, in the following publications: Current Protocols In Molecular Biology, supra, Chapter 8; Molecular Cloning: A Laboratory Manual., 2^{nd} edition (Sambrook et al., 1989); Zoller et al., Methods Enzmmol. 100:468-500 (1983); Zoller & Smith, DNA 3:479-488 (1984); Zoller et al., Nucl. Acids Res., 10:6487 (1987); Brake et al., Proc. Natl. Acad. Sci. USA 81:4642-4646 (1984); Botstein et al., Science 229:1193 (1985); Kunkel et al., Methods Enzymol. 154:367-82 (1987), Adelman et al, DNA 2:183 (1983); and Carter et al., Nucl. Acids Res., 13:4331 (1986). Cassette mutagenesis (Wells et al., Gene, 34:315 [1985]), and restriction selection mutagenesis (Wells et al., Philos. Trans. R. Soc. London SerA, 317:415 [1986]) may also be used.

Amino acid sequence variants with more than one amino acid substitution may be generated in one of several ways. If the amino acids are located close together in the polypeptide chain, they may be mutated simultaneously, using one oligonucleotide that codes for all of the desired amino acid substitutions. If, however, the amino acids are located some distance from one another (e.g. separated by more than ten amino acids), it is more difficult to generate a single oligonucleotide that encodes all of the desired changes. Instead, one of two alternative methods may be employed. In the first method, a separate oligonucleotide is generated for each amino acid to be substituted. The oligonucleotides are then annealed to the single-stranded template DNA simultaneously, and the second strand of DNA that is synthesized from the template will encode all of the desired amino acid substitutions. The alternative method involves two or more rounds of mutagenesis to produce the desired mutant.

The amino acid sequence variants of the present invention include polypeptides in which the membrane spanning (transmembrane) region or regions are deleted or inactivated. For example, in the rat polypeptide of SEQ ID NO:1 amino acids 35-55 and 123-143 have been tentatively identified as membrane spanning segments. Deletion or inactivation of these portions of the molecule yields soluble proteins, which are no longer capable of membrane anchorage. Inactivation may, for example, be achieved by deleting sufficient residues (but less than the entire transmembrane region) to produce a substantially hydrophilic hydropathy profile at this site, or by substituting with heterologous residues which accomplish the same result. For example, the transmembrane region(s) may be substituted by a random or predetermined sequence of about 5 to 50 serine, threonine, lysine, arginine, glutamine, aspartic acid and like hydrophilic residues, which altogether exhibit a hydrophilic hydropathy profile. Like the transmembrane region deletional variants, these variants are “soluble”, i.e. secreted into the culture medium of recombinant hosts. Soluble variants of the native polypeptides of the present invention may be used to make fusions at their N- or C-terminus to immunogenic polypeptides, e.g. bacterial polypeptides such as beta-lactamase or an enzyme encoded by the E. coli trp locus, or yeast protein, and C-terminal fusions with proteins having a long half-life such as immunoglobulin regions (preferably immunoglobulin constant regions to yield immunoadhesins), albumin, or ferritin, as described in WO 89/02922 published on 6 Apr. 1989. For the production of immunoglobulin fusions see also U.S. Pat. No. 5,428,130 issued Jun. 27, 1995.

Production of Antibodies

The present invention includes antibodies that specifically bind a polypeptide of SEQ ID NO:1 or another mammalian (e.g. human) homologue of such polypeptide. Such antibodies find utility as reagents used, for example, in analytical chemistry or process sciences, as diagnostic and/or therapeutics.

Methods of preparing polyclonal antibodies are known in the art. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. It may be useful to conjugate the immunizing agent to a protein known to be immunogenic in the mammal being immunized, such as serum albumin, or soybean trypsin inhibitor. Examples of adjuvants which may be employed include Freund's complete adjuvant and MPL-TDM.

According to one approach, monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. Preferred immortalized cell lines are those that fuse efficiently, support stable high level expression of antibody by the selected antibody-producing cells, and are sensitive to a medium such as HAT medium.

The culture medium in which the hybridoma cells are cultured can then be assayed for the presence of monoclonal antibodies directed against the particular polypeptide used, such as a rat polypeptide of SEQ ID NO:1 or its human homologue. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods [Goding, supra]. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Alternatively, monoclonal antibodies may be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells discussed above serve as a preferred source of such DNA. Once isolated, the DNA may be placed into expression vectors, which are then transfected into host cells such as COS cells, Chinese hamster ovary (CHO) cells, or myeloma cells that do not otherwise produce immunoglobulin protein, to obtain the synthesis of monoclonal antibodies in the recombinant host cells.

The antibodies, including antibody fragments, such as Fv, Fab, Fab′, F(ab′)₂ or other antigen-binding subsequences of antibodies, may be humanized. Humanized antibodies contain minimal sequence derived from a non-human immunoglobulin. More specifically, in humanized antibodies residues from a complementary determining region (CDR) of a human immunoglobulin (the recipient) are replaced by residues from a CDR of a non-human species (donor antibody) such as mouse, rat or rabbit having the desired specificity, affinity and capacity. In some instances, Fv framework residues of the human immunoglobulin are also replaced by corresponding non-human residues. Humanized antibodies may additionally comprise residues that are found neither in the recipient antibody nor in the imported CDR or framework sequences [Jones et al., Nature, 321:522-525 (1986); Riechmann et al., Nature, 332:323-329 (1988)].

Methods for humanizing non-human antibodies are well known in the art. Generally, a humanized antibody has one or more amino acid residues introduced into it from a non-human source. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. In addition, human antibodies can be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boemer et al. are also available for the preparation of human monoclonal antibodies (Cole et al., Monoclonal Antibodies and Cancer Therapy, Alan R. Liss, p. 77 (1985) and Boemer et al., J. Immunol., 147(1):86-95 (1991)]. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

The antibodies may be bispecific, in which one specificity is for polypeptide of the present invention, and the other specificity for another protein, such as, a second polypeptide of the present invention or another polypeptide.

4. Uses

a. Polynucleotides

The differentially expressed genes identified in accordance with the present invention may be used to design specific oligonucleotide probes and primers. In certain preferred embodiments, the term “primer” as used here includes any nucleic acid capable of priming template-dependent synthesis of a nascent nucleic acid. In certain other embodiments, the nucleic acid may be able to hybridize a template, but not be extended for synthesis of nascent nucleic acid that is complementary to the template.

In certain embodiments of the present invention the term “template” may refer to a nucleic acid that is used in the creation of a complementary nucleic acid strand to the “template” strand. The template may be either RNA or DNA, and the complementary strand may also be RNA or DNA. In certain embodiments the complementary strand may comprise all or part of the complementary sequence to the template, or may include mutations so that it is not an exact, complementary strand to the template. Strands that are not exactly complementary to the template strand may hybridize specifically to the template strand in detection assays described here, as well as other assays known in the art, and such complementary strands that can be used in detection assays are part of the invention.

When used in combination with nucleic acid amplification procedures, these probes and primers enable the rapid analysis of cell, tissue, or peripheral blood samples. In certain aspects of the invention, the term “amplification” may refer to any method or technique known in the art or described herein for duplicating or increasing the number of copies or amount of a target nucleic acid or its complement. The term “amplicon” refers to the target sequence for amplification, or that part of a target sequence that is amplified, or the amplification products of the target sequence being amplified. In certain other embodiments, an “amplicon” may include the sequence of probes or primers used in amplification. This analysis assists in detecting and diagnosing a disease, specifically cardiac, kidney or inflammatory disease, and in determining optimal treatment courses for individuals at varying stages of disease progression.

In light of the present disclosure, one skilled in the art may select segments from the identified genes for use in detection, diagnostic, or prognostic methods, vector constructs, antibody production, kits, or any of the embodiments described herein as part of the present invention. For example, in certain embodiments the sequences selected to design probes and primers may include repetitive stretches of adenine nucleotides (poly-A tails) normally attached at the ends of the RNA for the identified differentially expressed gene. In certain other embodiments, probes and primers may be specifically designed to not include these or other segments from the identified genes, as one of ordinary skill in the art may deem certain segments more suitable for use in the detection methods disclosed.

For example, where a genomic sequence is disclosed, one may use sequences that correspond to exon regions of the gene in most cases. One skilled in the art may select segments from the published exon sequences, or may assemble them into a reconstructed mRNA sequence that does not contain intronic sequences. Indeed, one skilled in the art may select or assemble segments from any of the identified gene sequences into other useful forms, such as coding segment reconstructions of mRNA sequences from published genomic sequences of the identified differentially expressed genes, as part of the present invention. Such assembled sequences would be useful in designing probes and primers, as well as providing coding segments for protein translation and for detection, diagnosis, and prognosis embodiments of the invention described herein.

Primers can be designed to amplify transcribed portions of the differentially expressed genes of the present invention that would include any length of nucleotide segment of the transcribed sequences, up to and including the full length of each gene. It is preferred that the amplified segments of identified genes be an amplicon of at least about 50 to about 500 base pairs in length. It is more preferred that the amplified segments of identified genes be an amplicon of at least about 100 to about 400 base pairs in length, or no longer in length than the amplified segment used to normalize the quantity of message being amplified in the detection assays described herein. Such assays include RNA diagnosticing methods, however, differential expression may be detected by other means, and all such methods would fall within the scope of the present invention. The predicted size of the gene segment, calculated by the location of the primers relative to the transcribed sequence, would be used to determine if the detected amplification product is indeed the gene being amplified. Sequencing the amplified or detected band that matches the expected size of the amplification product and comparison of the band's sequence to the known or disclosed sequence of the gene would confirm that the correct gene is being amplified and detected.

The identified differentially expressed genes may also be used to identify and isolate full-length gene sequences, including regulatory elements for gene expression, from genomic human DNA libraries. The cDNA sequences or portions thereof, identified in the present disclosure may be used as hybridization probes to screen genomic human (or other mammalian) DNA libraries by conventional techniques. Once partial genomic clones have been identified, “chromosomal walking” may isolate full-length genes (also called “overlap hybridization”). See Chinault et al., Gene 5:111-26 (1979). Once a partial genomic clone has been isolated using a cDNA hybridization probe, nonrepetitive segments at or near the ends of the partial genomic clone may be used as hybridization probes in further genomic library screening, ultimately allowing isolation of entire gene sequences for the disease, specifically cardiac, kidney or inflammatory disease, state genes of interest. It will be recognized that full-length genes may be obtained using small ESTs via technology currently available and described in this disclosure (Sambrook et al., supra; Chinault et al., supra). Sequences identified and isolated by such means may be useful in the detection of disease genes using the detection and diagnostic methods described herein, and are part of the invention.

As described before, the identified rat gene may be used as a hybridization probe to screen human or other mammalian cDNA libraries by conventional techniques. Comparison of cloned cDNA sequences with known human or animal cDNA or genomic sequences may be performed using computer programs and databases known in the art.

The polynucleotides of the present invention are also useful in antisense-mediated gene inhibition, first introduced by Stephenson and Zamecnik (Proc. Natl. Acad. Sci. USA 75:285-288 [1978]; see also, Zamecnik et al., Proc. Natl. Acad. Sci. USA 83, 4143-4146 [1986]). This technique is based on the discovery that synthetic DNA fragments can inhibit the transcription and/or translation of selected genes in a sequence-specific manner. Since its inception, the technique has found important diagnostic and clinical therapeutic applications in many fields of oncology, vascular and genetic diseases, and in the treatment of HIV and other virus infections. To date, two main antisense strategies have been employed: transfection of cells with antisense cDNA and treatment of cells with antisense oligodeoxynucleotides (ODNs), the use of ODNs derived from the translation initiation site, e.g., between the −10 and +10 regions of the target gene nucleotide sequence of interest being preferred. According to the present invention, molecules can be designed to reduce or inhibit either normal or, if appropriate, mutant target gene activity, using antisense technology. For further details see, for example, Wagner, “Gene inhibition using antisense oligodeoxynucleotides.” Nature 372:333-335 (1992); Tonkinson and Stein, “Antisense oligodeoxynucleotides as clinical therapeutic agents.” Cancer Invest. 14:54-65 (1996); Askari and McDonnell, “Antisense-oligonucleotide therapy.” N. Engl. J. Med. 334:316-318 (1996); Redekop and Naus, “Transfection with bFGF sense and antisense cDNA resulting in modification of malignant glioma growth.” J. Neurosura. 82:83-90 (1997); Saleh et al., “Inhibition of growth of C6 glioma cells in vivo by expression of antisense vascular endothelial growth factor sequence.” Cancer Res. 56:393-401 (1996).

Oligodeoxynucleotides can be used for the inhibition of gene transcription in the form of triple helix structures. The base composition of these oligodeoxynucleotides must be designed to promote triple helix formation via Hoogsteen base pairing rules, which generally require sizeable stretches of either purines or pyrimidines to be present on one strand of a duplex. Nucleotide sequences can be pyrimidine-based, which will result in TAT and CGC+triplets across the three associated strands of the resulting triple helix. The pyrimidine-rich molecules provide base complementarily to a purine-rich region of a single strand of the duplex, in a parallel orientation to that strand. In addition, nucleic acid molecules can be chosen that are purine-rich and, for example, contain a stretch of G residues. These molecules form a triple helix with a DNA duplex that is rich in GC pairs, in which the majority of the purine residues are located on a single strand of the targeted duplex, resulting in GGC triplets across the three strands in the triplex. Alternatively, creating a “switchback” nucleic acid molecule can increase the potential sequences that can be targeted for triple helix formation. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

The invention also covers the use of ribozymes. Ribozymes are enzymatic RNA molecules capable of catalyzing the specific cleavage of RNA (Rossi, Current Biology 4:469-71 [1994]). The mechanism of ribozyme action involves sequence specific hybridization of the ribozyme molecule to complementary target RNA, followed by an endonucleolytic cleavage. The composition of ribozyme molecules must include one or more sequences complementary to the target gene mRNA and must include the well-known catalytic sequence responsible for mRNA cleavage. For this sequence, see U.S. Pat. No. 5,093,246, which is incorporated by reference herein in its entirety. Within the scope of the present invention are engineered hammerhead motif ribozyme molecules that specifically and efficiently catalyze endonucleolytic cleavage of RNA sequences encoding target gene proteins.

Specific ribozyme cleavage sites within any potential RNA target are initially identified by scanning the molecule of interest for ribozyme cleavage sites which include the following sequences, GUA, GUU and GUC. Once identified, short RNA sequences of between 15 and 20 ribonucleotides corresponding to the region of the target gene containing the cleavage site can be evaluated for predicted structural features, such as secondary structure, that can render the oligonucleotide sequence unsuitable. The suitability of candidate sequences can also be evaluated by testing their accessibility to hybridization with complementary oligonucleotides, using ribonuclease protection assays.

In instances where the antisense, ribozyme, or triple helix molecules are utilized to reduce or inhibit mutant gene expression, it is possible that the transcription or translation of mRNA produced by normal alleles is also reduced or inhibited. As a result, the concentration of normal gene product may be lower than is necessary for a normal phenotype. In such cases, to ensure that substantially normal levels of gene activity are maintained, nucleic acid molecules that encode and express the polypeptide encoded by the gene targeted, can be introduced into cells via gene therapy methods, such as those described below. The nucleic acid sequence used in gene therapy is selected such that it does not contain sequences susceptible to the antisense, ribozyme, or triple helix treatments utilized. Alternatively, where the target gene encodes an extracellular protein, it can be preferable to co-administer normal target gene protein into the cell or tissue in order to maintain the requisite level of cellular or tissue target gene activity.

The present invention also contemplates the use of “peptide nucleic acids” (PNAs). PNAs have a peptide-like backbone instead of the normal sugar and phosphate groups of DNA. PNAs may be used to turn on specific genes, by binding to a promoter region of a gene to initiate RNA transcription. This approach is particularly useful where a particular disease or disorder is characterized by the underexpression of a particular gene, or where the increased expression of an identified gene has a beneficial effect on the treatment of a disease, in particular cardiac, kidney or inflammatory disease. Chimeric molecules of PNA and DNA may also be considered. The DNA portion will allow enzymes attacking DNA-RNA hybrids to cut the RNA part of the complex into pieces (leading to dissociation of the drug molecule, which can then be reused), whereas the PNA portion will contribute stability and selectivity.

As noted before, the polynucleotides of the present invention can also be used in gene therapy. In gene therapy applications, genes are introduced into cells in order to achieve in vivo synthesis of a therapeutically effective genetic product, for example for replacement of a defective gene. Gene therapy includes both conventional gene therapy where a lasting effect is achieved by a single treatment, and the administration of gene therapeutic agents, which involves the one time or repeated administration of a therapeutically effective DNA or RNA.

There are a variety of techniques available for introducing nucleic acid into viable cells. The techniques differ depending upon whether the nucleic acid in transferred into cultured cells in vitro, or in vivo in the cells of the intended host. Techniques suitable for the transfer of the nucleic acid into mammalian cells in vitro include the use of liposomes, electroporation, microinjection, cell fusion, DEAE-dextran, the calcium phosphate method, etc. The currently preferred in vivo gene transfer methods include transfection with viral (typically retroviral) vectors and viral coat protein-liposome mediated transfection (Dzau et al., Trends in Biotechnology 11, 205-210 [1993]). In some situations it is desirable to provide the nucleic acid source with an agent that targets the target cells, such as an antibody specific for a cell surface membrane protein or the target cells, a ligand for a receptor on the target cells, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. For review of gene marking and gene therapy protocols see Anderson et al, Science 256, 808-813 (1992).

The information provided by the present invention can also be used to detect genetic lesions in a differentially expressed gene of the present invention, thereby determining if a subject with the lesioned gene is at risk for a disorder characterized by differentially expressed gene expression or polypeptide activity. In preferred embodiments, the methods include detecting, in a biological sample from a subject, the presence or absence of a genetic lesion characterized by, for example, an alteration affecting the integrity of a gene encoding an polypeptide or the misexpression of the gene. For example, such genetic lesions can be detected by ascertaining the existence of at least one of: a deletion of one or more nucleotides from a gene; an addition of one or more nucleotides to a gene; a substitution of one or more nucleotides of a gene; a chromosomal rearrangement of a gene; an alteration in the level of a messenger RNA transcript of a gene; aberrant modification of a gene, such as of the methylation pattern of the genomic DNA; the presence of a non-wild type splicing pattern of a messenger RNA transcript of a gene; a non-wild type level of a gene protein; allelic loss of a gene; and inappropriate post-translational modification of a gene protein. As described herein, there are a large number of assay techniques known in the art that can be used for detecting lesions in a gene.

In certain embodiments, detection of a lesion may involve the use of a probe/primer in, such as anchor PCR or RACE PCR, or, alternatively, in LCR (see, e.g., Landegran et al., Science 241: 1077-80 [1988]; and Nakazawa et al., Proc. Natl. Acad. Sci. USA 91: 360-64 [1994]), the latter of which can be particularly useful for detecting point mutations in the cardiac gene (see Abravaya et al., Nucleic Acids Res. 23: 675-82 [1995]). This method can include the steps of collecting a biological sample from a subject, isolating nucleic acid (e.g., genomic, mRNA or both) from the cells of the sample, contacting the nucleic acid sample with one or more primers which specifically hybridize to an differentially expressed gene under conditions such that hybridization and amplification of the cardiac gene (if present) occurs, and detecting the presence or absence of an amplification product, or detecting the size of the amplification product and comparing the length to a control sample.

In an alternative embodiment, mutations in a differentially expressed gene from a sample can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see U.S. Pat. No. 5,498,531) can be used to score for the presence of specific mutations by development or loss of a ribozyme cleavage site.

The arrays of immobilized DNA fragments may also be used for genetic diagnostics. To illustrate, a microarray containing multiple forms of a mutated gene or genes can be probed with a labeled mixture of a subject DNA, which will preferentially interact with only one of the immobilized versions of the gene.

The detection of this interaction can lead to a medical diagnosis. Arrays of immobilized DNA fragments can also be used in DNA probe diagnostics. For example, the identity of a differentially expressed gene of the present invention can be established unambiguously by hybridizing a sample of a subject's DNA to an array comprising known differentially expressed DNA. Other molecules of genetic interest, such as cDNAs and RNAs can be immobilized on the array or alternately used as the labeled probe mixture that is applied to the array.

b. Polypeptides

The polypeptides of the present invention, including the polypeptides of SEQ ID NO:1 and it equivalents in other mammalian (e.g. human) species, can be used to identify interacting proteins and genes encoding such proteins. Interacting proteins and their genes may be part of the signaling pathway in which the differentially expressed genes identified herein participate, and thus are valuable diagnostic and therapeutic candidates or targets. Among the traditional methods employed are co-immunoprecipitation, cross-linking and co-purification through gradients or chromatographic columns. Using procedures such as these allows for the identification of interactive gene products. Once identified, an interactive gene product can be used, using standard techniques, to identify its corresponding interactive gene. For example, at least a portion of the amino acid sequence of the interactive gene product can be ascertained using techniques well known to those of skill in the art, such as the Edman degradation technique (see, e.g., Creighton, Proteins: Structures and Molecular Principles, W. H. Freeman & Co. (New York, N.Y. [1983], pp. 34-49). The amino acid sequence obtained can be used as a guide for the generation of oligonucleotide mixtures that can be used to screen for interactive gene sequences. Screening can be accomplished, for example, by standard hybridization or PCR techniques. Techniques for the generation of oligonucleotide mixtures and the screening are well known.

Additionally, methods can be employed which result in the simultaneous identification of interactive genes that encode the protein interacting with a protein involved in a disease, specifically cardiac, kidney or inflammatory disease. These methods include, for example, probing expression libraries with a labeled protein known or suggested to be involved in a disease, using this protein in a manner similar to the well known technique of antibody probing of λgtll libraries.

A particularly suitable technique for studying protein-protein interactions is the yeast two-hybrid assay. Many transcriptional activators, such as yeast GALA, consist of two physically discrete modular domains, one acting as the DNA-binding domain, while the other one functioning as the transcription activation domain. The yeast two-hybrid system takes advantage of this property, and employs two hybrid proteins, one in which the target protein is fused to the DNA-binding domain of GAL4, and another, in which candidate activating proteins are fused to the activation domain. The expression of a GAL1-calZ reporter gene under control of a GAL4-activated promoter depends on reconstitution of GAL4 activity via protein-protein interaction. Colonies containing interacting polypeptides are detected with a chromogenic substrate for β-galactosidase. A complete kit (MATCHMAKER™) for identifying protein-protein interactions using the yeast two-hybrid technique is available from Clontech. For further details see e.g. Fields and Song, Nature (London) 340:245-246 (1989); Chien et al., Proc. Natl. Acad. Sci. USA 88:9578-9582 (1991); and Chevray and Nathans, Proc. Natl. Acad. Sci. USA 89:5789-5793 (1992).

Polypeptides of the present invention may also be used to generate antibodies, using well-known techniques, some of which have been detailed above.

The polypeptides of the present invention are also useful in assays for identifying lead compounds for therapeutically active agents for the treatment of cardiac, kidney or inflammatory diseases. Candidate compounds include, for example, peptides such as soluble peptides, including Ig-tailed fusion peptides (e.g. immunoadhesins) and members of random peptide libraries (see, e.g., Lam et al., Nature 354:82-84 (1991); Houghten et al., Nature 354:84-86 (1991)) and combinatorial chemistry-derived molecular libraries made of D- or L-configuration amino acids; phosphopeptides (e.g., members of random and partially degenerate, directed phosphopeptide libraries, see, e.g., Songyang et al., Cell 72:767-78 (1993); antibodies (e.g., polyclonal, monoclonal, humanized, anti-idiotypic, chimeric, and single chain antibodies as well as Fab, F(ab′)2, Fab expression library fragments, and epitope-binding fragments of antibodies); and small organic and inorganic molecules (e.g., molecules obtained from combinatorial and natural product libraries).

Such screening assays are preferably amenable to high-throughput screening of chemical libraries, and are particularly suitable for identifying small molecule drug candidates. Small molecules, which are usually less than 10K molecular weight, are desirable as therapeutics since they are more likely to be permeable to cells, are less susceptible to degradation by various cellular mechanisms, and are not as apt to elicit immune response as proteins. Small molecules include but are not limited to synthetic organic or inorganic compounds, and peptides. Many pharmaceutical companies have extensive libraries of such molecules, which can be conveniently screened by using the assays of the present invention. the assays can be performed in a variety of formats, including protein-protein binding assays, biochemical screening assays, immunoassays, cell based assays, etc. Such assay formats are well known in the art.

In a preferred embodiment, the screening assays of the present invention involve contacting a biological sample obtained from a subject having a disease, specifically cardiac, kidney or inflammatory disease, characterized by the differential expression of a gene identified herein, with a candidate compound or agent. The expression of the gene or the activity of the gene product is then determined in the presence and absence of the test compound or agent. When expression of differentially expressed gene mRNA or polypeptide is greater (preferably statistically significantly greater) in the presence of the candidate compound than in its absence, the candidate compound may be identified as a stimulator of differentially expressed gene expression. Alternatively, when differentially expressed gene expression is less (preferably statistically significantly less) in the presence of the candidate compound than in its absence, the candidate compound may be identified as an inhibitor of differentially expressed gene expression. The level of differentially expressed gene expression in the cells can be determined by methods described herein for detecting differentially expressed gene mRNA or protein.

Compounds identified via assays such as those described herein can be useful, for example, in elaborating the biological function of the target gene product, and for treating a cardiac, kidney or inflammatory disease, or ameliorating symptoms of such disease. In instances when a disease state or disorder results from a lower overall level of target gene expression, target gene product, or target gene product activity in a cell involved in the disease, compounds that interact with the target gene product can include ones accentuating or amplifying the activity of the bound target gene protein. Such compounds would bring about an effective increase in the level of target gene activity, thus treating the disease, disorder or state, or ameliorating its symptoms. Where mutations within the target gene cause aberrant target gene proteins to be made, which have a deleterious effect that leads to a disease, compounds that bind target gene protein can be identified that inhibit the activity of the bound target gene protein.

5. Pharmaceutical Compositions

Pharmaceutical compositions of the present invention can comprise a polynucleotide of the present invention, a product of the genes identified herein, or other therapeutically active compounds, including organic small molecules, peptides, polypeptides, antibodies etc. identified with the aid of the differentially expressed genes identified herein.

Suitable forms, in part, depend upon the use or the route of entry, for example oral, transdermal, inhalation, or by injection. Such forms should allow the agent or composition to reach a target cell whether the target cell is present in a multicellular host or in culture. For example, pharmacological agents or compositions injected into the blood stream should be soluble. Other factors are known in the art, and include considerations such as toxicity and forms that prevent the agent or composition from exerting its effect.

The active ingredient, when appropriate, can also be formulated as pharmaceutically acceptable salts (e.g., acid addition salts) and/or complexes. Pharmaceutically acceptable salts are non-toxic at the concentration at which they are administered. Pharmaceutically acceptable salts include acid addition salts such as those containing sulfate, hydrochloride, phosphate, sulfonate, sulfamate, sulfate, acetate, citrate, lactate, tartrate, methanesulfonate, ethanesulfonate, benzenesulfonate, p-toluenesulfonate, cyclolexylsulfonate, cyclohexylsulfamate and quinate. Pharmaceutically acceptable salts can be obtained from acids such as hydrochloric acid, sulfuric acid, phosphoric acid, sulfonic acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfonic acid, cyclohexylsulfamic acid, and quinic acid. Such salts may be prepared by, for example, reacting the free acid or base forms of the product with one or more equivalents of the appropriate base or acid in a solvent or medium in which the salt is insoluble, or in a solvent such as water which is then removed in vacuo or by freeze-drying or by exchanging the ions of an existing salt for another ion on a suitable ion exchange resin.

Carriers or excipients can also be used to facilitate administration of the compound. Examples of carriers and excipients include calcium carbonate, calcium phosphate, various sugars such as lactose, glucose, or sucrose, or types of starch, cellulose derivatives, gelatin, vegetable oils, polyethylene glycols and physiologically compatible solvents. The compositions or pharmaceutical composition can be administered by different routes including, but not limited to, intravenous, intra-arterial, intraperitoneal, intrapericardial, intracoronary, subcutaneous, and intramuscular, oral, topical, or transmucosal.

The desired isotonicity of the compositions can be accomplished using sodium chloride or other pharmaceutically acceptable agents such as dextrose, boric acid, sodium tartrate, propylene glycol, polyols (such as mannitol and sorbitol), or other inorganic or organic solutes.

Pharmaceutical compositions can be formulated for a variety of modes of administration, including systemic and topical or localized administration. Techniques and formulations generally may be found in Remington 's Pharmaceutical Sciences, 18^{th} Edition, Mack Publishing Co., Easton, Pa. 1990. See, also, Wang and Hanson “Parenteral Formulations of proteins and Peptides: Stability and Stabilizers”, Journal of Parenteral Science and Technology, Technical Report No. 10, Supp. 42-2S (1988). A suitable administration format can best be determined by a medical practitioner for each patient individually.

For systemic administration, injection is preferred, e.g., intramuscular, intravenous, intra-arterial, intracoronary, intrapericardial, intraperitoneal, subcutaneous, intrathecal, or intracerebrovascular. For injection, the compounds of the invention are formulated in liquid solutions, preferably in physiologically compatible buffers such as Hank's solution or Ringer's solution. Alternatively, the compounds of the invention are formulated in one or more excipients (e.g., propylene glycol) that are generally accepted as safe as defined by USP standards. They can, for example, be suspended in an inert oil, suitably a vegetable oil such as sesame, peanut, olive oil, or other acceptable carrier. Preferably, they are suspended in an aqueous carrier, for example, in an isotonic buffer solution at pH of about 5.6 to 7.4. These compositions can be sterilized by conventional sterilization techniques, or can be sterile filtered. The compositions can contain pharmaceutically acceptable auxiliary substances as required to approximate physiological conditions, such as pH buffering agents. Useful buffers include for example, sodium acetate/acetic acid buffers. A form of repository or “depot” slow release preparation can be used so that therapeutically effective amounts of the preparation are delivered into the bloodstream over many hours or days following transdermal injection or delivery. In addition, the compounds can be formulated in solid form and redissolved or suspended immediately prior to use. Lyophilized forms are also included.

Alternatively, certain compounds identified in accordance with the present invention can be administered orally. For oral administration, the compounds are formulated into conventional oral dosage forms such as capsules, tablets and tonics.

Systemic administration can also be by transmucosal or transdermal. For transmucosal or transdermal administration, penetrants appropriate to the barrier to be permeated are used in the formulation. Such penetrants are generally known in the art, and include, for example, for transmucosal administration, bile salts and fusidic acid derivatives. In addition, detergents can be used to facilitate permeation. Transmucosal administration can be, for example, through nasal sprays or using suppositories.

For administration by inhalation, usually inhalable dry power compositions or aerosol compositions are used, where the size of the particles or droplets is selected to ensure deposition of the active ingredient in the desired part of the respiratory tract, e.g. throat, upper respiratory tract or lungs. Inhalable compositions and devices for their administration are well known in the art. For example, devices for the delivery of aerosol medications for inspiration are known. One such device is a metered dose inhaler that delivers the same dosage of medication to the patient upon each actuation of the device. Metered dose inhalers typically include a canister containing a reservoir of medication and propellant under pressure and a fixed volume metered dose chamber. The canister is inserted into a receptacle in a body or base having a mouthpiece or nosepiece for delivering medication to the patient. The patient uses the device by manually pressing the canister into the body to close a filling valve and capture a metered dose of medication inside the chamber and to open a release valve which releases the captured, fixed volume of medication in the dose chamber to the atmosphere as an aerosol mist. Simultaneously, the patient inhales through the mouthpiece to entrain the mist into the airway. The patient then releases the canister so that the release valve closes and the filling valve opens to refill the dose chamber for the next administration of medication. See, for example, U.S. Pat. No. 4,896,832 and a product available from 3M Healthcare known as Aerosol Sheathed Actuator and Cap.

Another device is the breath actuated metered dose inhaler that operates to provide automatically a metered dose in response to the patient's inspiratory effort. One style of breath actuated device releases a dose when the inspiratory effort moves a mechanical lever to trigger the release valve. Another style releases the dose when the detected flow rises above a preset threshold, as detected by a hot wire anemometer. See, for example, U.S. Pat. Nos. 3,187,748; 3,565,070; 3,814,297; 3,826,413; 4,592,348; 4,648,393; 4,803,978.

Devices also exist to deliver dry powdered drugs to the patient's airways (see, e.g. U.S. Pat. No. 4,527,769) and to deliver an aerosol by heating a solid aerosol precursor material (see, e.g. U.S. Pat. No. 4,922,901). These devices typically operate to deliver the drug during the early stages of the patient's inspiration by relying on the patient's inspiratory flow to draw the drug out of the reservoir into the airway or to actuate a heating element to vaporize the solid aerosol precursor.

Devices for controlling particle size of an aerosol are also known, see, for example, U.S. Pat. Nos. 4,790,305; 4,926,852; 4,677,975; and 3,658,059.

For topical administration, the compounds of the invention are formulated into ointments, salves, gels, or creams, as is generally known in the art.

If desired, solutions of the above compositions can be thickened with a thickening agent such as methyl cellulose. They can be prepared in emulsified form, either water in oil or oil in water. Any of a wide variety of pharmaceutically acceptable emulsifying agents can be employed including, for example, acacia powder, a non-ionic surfactant (such as a Tween), or an ionic surfactant (such as alkali polyether alcohol sulfates or sulfonates, e.g., a Triton).

Compositions useful in the invention are prepared by mixing the ingredients following generally accepted procedures. For example, the selected components can be mixed simply in a blender or other standard device to produce a concentrated mixture which can then be adjusted to the final concentration and viscosity by the addition of water or thickening agent and possibly a buffer to control pH or an additional solute to control tonicity.

The amounts of various compounds for use in the methods of the invention to be administered can be determined by standard procedures. Generally, a therapeutically effective amount is between about 100 mg/kg and 10-12 mg/kg depending on the age and size of the patient, and the disease or disorder associated with the patient. Generally, it is an amount between about 0.05 and 50 mg/kg of the individual to be treated. The determination of the actual dose is well within the skill of an ordinary physician.

The invention is further illustrated in the following non-limiting examples.

### EXAMPLES

### Example 1

Identification of Differentially Expressed Rat Genes Referred to By Clone ID Number P00210 D09

1. In Vivo Model Of myocardial Infarction

Genes P00210_D09 was identified by analysis of left ventricular heart tissue obtained from an in vivo model of left ventricle myocardial infarction (MI) (Pfeffer et al., Circ. Res. 57:84-95 [1985]). Specifically, male Sprague-Dawley rats at age 7-10 weeks were anesthetized with ketamine (80 mg/kg IP) and xylazine (10 mg/kg IP). The thorax and abdomen was shaved, after which the areas were scrubbed with providone-iodine and 70% isopropyl alcohol a minimum of three times, beginning at the incision line and continuing in a circular motion proceeding toward the periphery. The rats were intubated and placed on a respirator with room air at a rate of 55 breaths/min. A left thoracotomy was performed between the fourth and fifth ribs, after which the heart was exteriorized and the left anterior descending coronary artery (LAD) ligated with silk suture. The same surgical procedure was employed for sham-operated rats, however, the suture was passed through the left ventricular wall and the LAD was not occluded.

Following the surgical procedure, negative pressure in the thoracic was quickly reestablished and the wound closed with a purse-string suture using 3-0 non-absorbable suture material. Butorphanoll (0.1 mg/kg. SQ) was provided post surgery as a prophylactic analgesic. The rats were extubated when they recovered their gag reflex and allowed recovering in a warming chamber. Seventy-five percent of the rats had large infarcts on their left ventricle free walls and perioperative mortality rate is about 50%, which is comparable to the published data.

Tissue was collected 2 week, 4 week, 8 week, 12 week and 16 week post-surgery. Blood was collected the day before surgery and the day before sacrifice for measurement of plasma atrial natriuretic peptide (ANP) level. On the day of necropsy, each heart was divided transversely into two halves so that the infarcted area is bisected. One half of the heart was used for histological evaluation, and the other for mRNA microarray analysis.

2. In vivo Model of Septum Myocardial Infarction

Septum tissue was obtained from diseased rat hearts obtained through the left ventricle rat MI model of Pfeffer et al., as described above. Poly A+ mRNA was prepared from each of these septums for assessment of differentially expressed genes in the disease state, using microarray analysis in a preferred embodiment.

3. Preparation of normalized cDNA libraries

Poly A+ mRNA was prepared from each of the animals, for assessment of differentially expressed genes in the disease state, using microarray analysis. Total RNA was isolated from homogenized tissue by acid phenol extraction (Chomczynski and Sacchi, Anal. Biochem. 162(1):156-9 [1987]). Poly A+ mRNA was selected from total RNA by oligo dT hybridization utilizing a polyA Spin mRNA Isolation Kit (New England BioLabs, Beverly, Mass.) according to manufacturers' protocols. A directionally cloned cDNA library was first generated by conventional methods. Briefly, double stranded cDNA was generated by priming first strand synthesis for reverse transcription using oligo dT primers which contain a Not I restriction site. After second strand synthesis, Xba I adapters were added to the 5′ end of the cDNA, and the cDNA size was selected for >500 bp and ligated into the corresponding restriction sites of phagemid vector pCR2.1 (Invitrogen, San Diego Calif.).

From the total cDNA library, a normalized library was generated as detailed elsewhere (see, e.g. Bonaldo et al., Genome Res. 6(9):791-806 [1996]) and described here briefly. Phagemid vector pCR2.1 contains an F1 origin of replication. Thus, the cDNA library can be propagated as single stranded phage with an appropriate helper virus. Single stranded, circular DNA was extracted from the phage library and served as “tester” DNA in the hybridization step of normalization. The other component of the hybridization, “driver” DNA, was generated from the library by PCR amplification using a set of the following primers specific for the region of the vector, which flanks the cloned inserts:

| | |
|---|---|
| 5′CGTATGTTGTGTGGAATTGTGAGCG | (SEQ ID NO:3) |
| 5′GATGTGCTGCAAGGCGATTAAGTTG | (SEQ ID NO:4) |

Purified tester DNA (50 ng) and driver DNA (0.5 μg) were combined in 120 mM NaCl, 50% formamide, 10 mM Tris (pH 8.0), 5 mM EDTA, and 1% SDS. A set of oligonucleotides (10 μg each), corresponding to polylinker sequence (same strand as tester DNA) which is present in the PCR product, was included in the hybridization reaction to block annealing of vector-specific sequences which are in common between tester and driver DNA. The oligonucleotide sequences were as follows:

| | |
|---|---|
| 5′GCCGCCAGTGTGCTGGAATTCGGCTAGC | (SEQ ID NO:5) |
| 5′CGAATTCTGCAGATATCCATCACACTGG | (SEQ ID NO:6) |
| 5′CTAGAGGGCCCAATTCGCCCTATAG | (SEQ ID NO:7) |
| 5′TGAGTCGTATTACAATTCACTGGCC | (SEQ ID NO:8) |
| 5′GCTCGGATCCACTAGTAACG | (SEQ ID NO:9) |
| 5′TTTTTTTTTTTTTTTTTT | (SEQ ID NO:10) |

The reaction mixture, under oil, was heated 3 min. at 80° C., and hybridization performed at 30° C. for 24 hr (calculated Cot ˜5). Single stranded circles were purified from the reaction mixture by hydroxylapatite (HAP) chromatography, converted to double strand DNA, and electroporated into bacteria to yield a normalized cDNA library representative of genes expressed in the left ventricle of rat. To evaluate the effectiveness of the normalization protocol, the frequency of a few clones (ANP, BNP, actin, and myosin) was assessed in both in the starting library and the normalized library. The frequency of abundant cDNAs (actin and myosin) was reduced and roughly equivalent to rarer cDNA clones (ANP and BNP). Clone frequency in the two libraries was determined with standard screening techniques by immobilizing colonies onto nylon membranes and hybridizing with radiolabeled DNA probes.

Certain genes, unexpressed in a normal tissue and turned on in diseased tissue, may be absent from the normalized cDNA library generated from normal tissue. To obtain disease-specific clones to include on the microarray, one can repeat the normalization strategy using diseased tissue obtained from the appropriate disease model. However, since most genes are expressed commonly between normal and diseased tissue, microarraying normalized libraries from diseased and normal tissue may introduce significant redundancy, a subtracted library can be made using protocols similar to those used to generate normalized libraries. Again, the method of Bonaldo et al., supra, as described here briefly, is used.

To make a subtracted library, a total cDNA library is generated from the tissue obtained from the disease model (e.g., left ventricle taken from the MI Model). The cDNA library is directionally cloned in pCR2.1 vector and single stranded tester DNA derived as described above for library normalization. The driver DNA is generated by PCR amplification of cloned inserts from the total cDNA library prepared from the left ventricle of normal rat. Hybridization occurs between sequences, which are in common to normal and diseased hearts. For this subtracted library, the reaction is driven more thoroughly (calculated Cot 27) than normalization by using more driver (1.5 μg vs. 0.5 μg) and longer hybridization time (48 hr vs. 24 hr). Purification of nonhybridized, single stranded circles by HAP chromatography, conversion to double strand DNA, and electroporation into bacteria yields a subtracted cDNA library enriched for genes which are expressed in diseased rat hearts. To test that the library is truly subtracted, colony hybridization is performed with probes for ANP, BNP, actin, and myosin. The subtracted library has a high frequency of ANP and BNP clones since they are elevated significantly in the hypertrophic rat heart. Actin and myosin clones are absent since they are expressed equally in normal and diseased left ventricle.

4. Microarray Analysis

High quality DNA is important for the microarray printing process. A microtiter plate protocol for PCR amplification of DNA and its subsequent purification was established that provides acceptable quality and quantity of DNA for printing on microarrays. Specifically, the following PCR probes were synthesized that amplify insert DNA from the vector pCR2.1 that was used for library construction:

| | |
|---|---|
| 5′CGTATGTTGTGTGGAATTGTGAGCG | (SEQ ID NO:11) |
| 5′GATGTGCTGCAAGGCGATTAAGTTG | (SEQ ID NO:12) |

After 30 cycles of amplification each PCR product was passed over a gel filtration column to remove unincorporated primers and salts. To maintain robustness, the columns were packed in 96-well filter plates and liquid handling was performed using a robotic liquid handler (Biomek 2000, Beckman).

To test the quality of DNA prepared by this PCR method, 96 purified samples from a single microtiter plate were produced as a microarray. Using the robotic liquid handler, 85 l of PCR reaction mixture was aliquoted into each well of a thin walled, 0.2 ml 96-well plate. The reaction mixture contained 0.2 mM each dNTP, 1.25 units of Taq polymerase, and 1×Taq buffer (Boehringer Mannheim). Primers, 1 μm each, are from vector regions, which flank the cloning site of pCR2.1 and include a 5′ primary amine with a 6-carbon linker to facilitate attachment of DNA product to the glass surface of the microarray chip. 1.0 μl of bacterial culture of individual cDNA clones was added to each well. PCR conditions were: 2 min., 95° C. to denature, then 30 cycles of 95° C., 30 sec./65° C., 40 sec./72° C., 1 min. 30 sec., and a final extension of 72° C., 5 min. using a MJResearch PTC 100 thermocycler.

PCR products were purified by gel filtration over Sephacryl 400 (Sigrna). Briefly, 400 μl of pre-swollen Sephacryl 400 was loaded into each well of a 96-well filter plate (PallBiosupport) and spun into a collection plate at 800 g for 1 min. Wells were washed 5 times with 0.2×SSC. PCR reaction mixtures were loaded onto the column and purified DNA (flow-through) was collected at 800 g for 1 min. Samples were dried down at 50° C. overnight and arrayed.

Fluorescent probe pairs were synthesized by reverse transcription of poly A+ RNA using, separately, Cy3 dCTP and Cy5 dCTP (Amersham). In 16.5 ll, 1 μg poly A+ RNA and 2 μg of oligo dT 21 mer, were denatured at 65° C., 5 min. and annealed at 25° C., 10 min. Reverse transcription was performed for 2 hours at 37° C. with Superscript RT (Life Technologies, Gaithersburg, Md.) in 1× buffer, 10 units RNase block, 500 μM each dATP/dGTP/dTTP, 280 μM dCTP, 40 μM Cy5 or Cy3 dCTP, and 200 units RT. RNA is degraded in 0.1 M NaOH, 65° C. for 10 min. Labeled cDNA was purified by successive filtration with Chroma Spin 30 spin columns (Clontech) following manufacturer's instructions. Samples were dried at room temperature in the dark using a covered Speed-Vac. Probes were applied to the test chip for hybridization and the data collected essentially as described in Schena et al., cited above The intensity of hybridization signal at each element reflected the level of expression of the mRNA for each gene in the rat ventricle. Digitized signal data was stored and prepared for analysis.

A series of control DNA elements were included on each chip to ensure consistency in labeling and hybridization between experiments and to aid in balancing the signal when two fluorescence channels are used. For each element hybridized with dual labeled probes, absolute and relative intensity of signal was determined. The results from these and other experiments indicate that these methods for production of template DNA and labeled cDNA probes are suitable for generating high quality microarrays within a preferred embodiment of the methods of the present invention. The evaluation of tens of thousands of genes for expression generates a large amount of data that can be manipulated by commercially available software packages that facilitate handling this type and quantity of data. The expression data can be stored, analyzed, and sorted from each experiment using this software. In addition, expression of each clone can be tracked from experiment to experiment using known methodologies.

The novel secreted factor of the present invention was identified from expression data from the following experiments: A 10,000 clone microarray (10K) from a normalized normal rat left ventricle (LV) cDNA library was probed in duplicate. A 3,000 clone array, which included differentially expressed clones from the 10K library, was also probed in duplicate. Included on the microarray with the unidentified genes were a set of known clones. These known clones were included because they represent genes of particular interest and help evaluate the sensitivity of the microarray methodology. Indeed, any genes of particular interest may be included on such microarrays. By way of example, ANP, BNP, endothelin, β-myosin heavy chain, and α-actin are genes that change expression levels in the LVH model, and thus they serve as useful positive controls in the in vivo model exemplified herein.

The intensity of hybridization signal at each element of the microarray reflected the level of expression of the mRNA for each gene. For each element hybridized with dual labeled probes, absolute and relative intensity of signal was determined, which translates into the relative expression levels of the subject genes. The numeric data obtained reflect the relative expression level of the gene in the disease state as compared to the expression level of the gene in the normal, or non-disease state. Positive numbers are indicative of genes expressed at higher levels in the diseased tissue relative to normal tissue, and negative values are indicative of lower expression in disease. Data are the average values from multiple experiments performed with separate DNA arrays (n=4 for MI left ventricle and septum). Array probes were generated from RNA pooled from multiple animals (n=4 for MI).

The data also reflect expression levels of genes in certain disease models over various time points. For example, gene expression in the myocardial infarction model was compared at 2, 4, 8, 12, and 16 weeks for the representative genes in the disease state versus the normal state. Indeed, such experimentation provides valuable data regarding the temporal relationship of gene expression levels in disease states and provides important insights regarding the treatment, diagnosis, and modulation of differentially expressed disease state genes, as discussed in detail infra.

One to two percent of the clones assayed on microarrays were found to be differentially expressed. Secondary chips may be used for more extensive hybridizations, including examination of individual animals, and more thorough evaluation of time points. In a preferred embodiment, clones that reproducibly scored in microarray analysis to be at least about 1.8-fold elevated or decreased were microarrayed on separate secondary chips and their expression levels determined. It is understood, however, that differentially expressed genes exhibiting less than about a two-fold change in expression, e.g., less than one, one-half, or one-quarter, or greater than about a two-fold change in expression, e.g., greater than three, five, ten, twenty, one hundred-fold, or one thousand-fold, are within the scope of the present invention.

5. Microarray Results

Using the foregoing protocols, it was found that in the MI model, the expression level of the gene corresponding to the clone referred to as P00210_D09 was 2.1-fold higher at the week 12 time point in the rat left ventricle and 1.8 fold higher at the 2 week time point in the septum. This overexpression suggests the possible involvement of this gene in the development and/or progress of MI.

6. Sequence Analysis

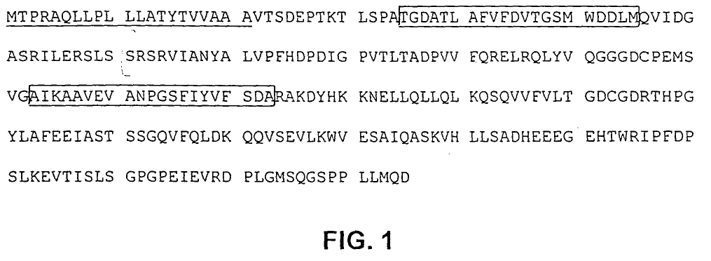

The differentially expressed partial and full-length clone P00210_D09 was sequenced (SEQ ID NO:2), and the deduced amino acid sequence was determined (SEQ ID NO:1). FIG. 1 show the deduced amino acid sequence of the polypeptide encoded by the clone P00210_D09. The approximate molecular weight of the polypepti8de is 29951.06 daltons, its isoelectric point is 4.606, and its charge at pH 7.0 is −14.114. Melting temperature (Davis, Botstein, Roth): 89.10° C. The open reading fram (ORF) of the polypeptide contains 275 amino acid residues, of which approximately the first 21 residues, including the initiating methionine, show the characteristics of a putative signal sequence, which is underlined in FIG. 1. The sequence includes two putative membrane-spanning segments at positions 35-55 and 123-143, respectively, which are boxed in the sequence. Of the 275 amino acids, 21 are strongly basic (+) (K, R), 36 are strongly acidic (−) (D, E), 103 are hydrophobic amino acids (A, I, L, F, W, V), and 67 are polar amino acids (N, C, Q, S, T, Y).

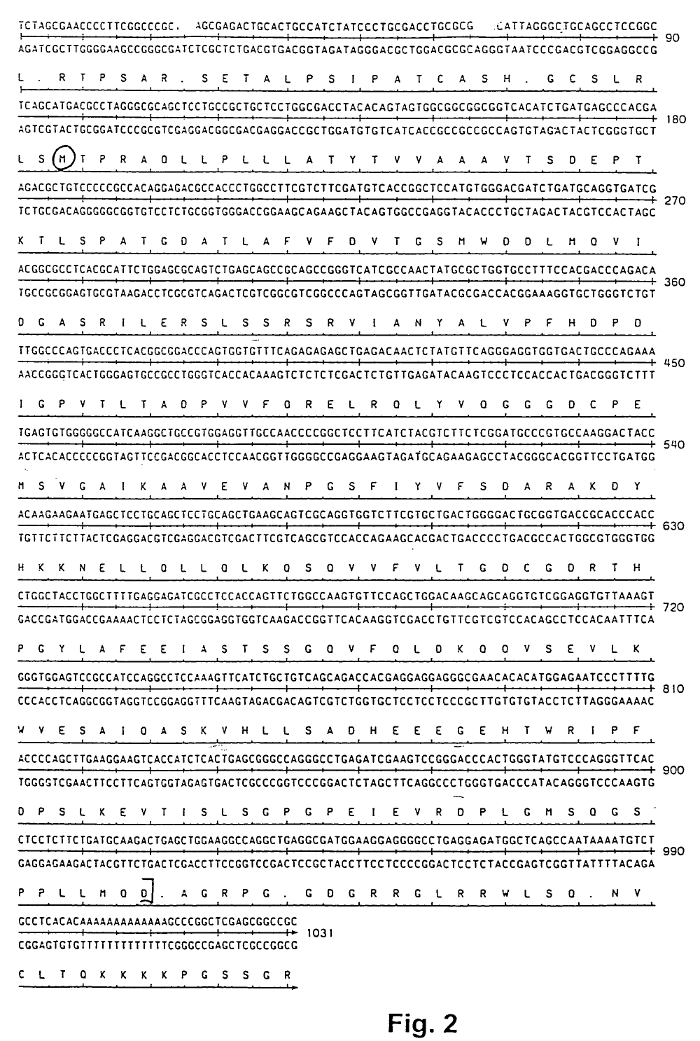

FIG. 2 (SEQ ID NO:2) shows that nucleotide sequence of the clone P00210_D09. The total length of this sequence is 1031 bases.

The nucleotide sequences of P00210_D09 was compared with sequences in the public GenBank, EMBL, DDBJ, PDB and GENSEQ databases. The search was performed using the BLASTN 2.0.8 program with default parameters. Gap penalties: existence: 5; extension: 2. The search revealed no significant homology with sequences present in the searched databases.

7. Northern Blot Analysis

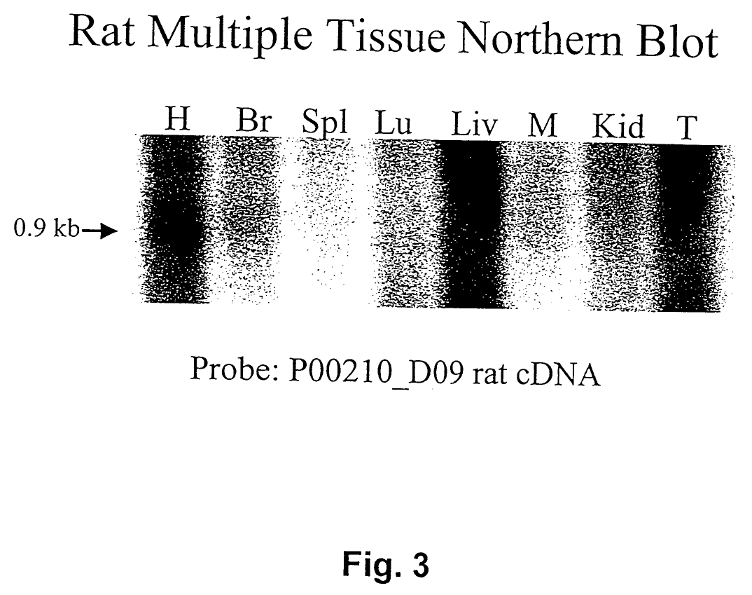

Northern blot analysis suggested that P00210 D09 encodes a rare message. A putative about 900 bp transcript was detected in rat heart using polyA+ mRNA (see FIG. 3).

### Example 2

Identification of the Human Homologue of Rat Clone

The isolated differentially expressed rat P00210_D09 gene sequence can be labeled and used to screen a cDNA library constructed from mRNA obtained from an organism of interest. Hybridization conditions will be of a lower stringency when the cDNA library was derived from an organism different from the type of organism from which the labeled sequence was derived. Alternatively, the labeled fragment can be used to screen a genomic library derived from the organism of interest, again, using appropriately stringent conditions. Such low stringency conditions will be well known to those of skill in the art, and will vary predictably depending on the specific organisms from which the library and the labeled sequences are derived. For guidance regarding such conditions see, Sambrook et al., supra, and Ausubel et al., supra.

PCR technology can also be utilized to isolate full-length human cDNA sequences. For example, RNA can be isolated, following standard procedures, from an appropriate human cellular or tissue source. A reverse transcription reaction can be performed on the RNA using an oligonucleotide primer specific for the most 5′ end of the amplified fragment for the priming of first strand synthesis. The resulting RNA/DNA hybrid can then be “tailed” with guanines using a standard terminal transferase reaction, the hybrid can be digested with RNase H, and second strand synthesis can then be primed with a poly-C primer. Thus, cDNA sequences upstream of the amplified fragment can easily be isolated. For a review of cloning strategies that can be used, see, e.g., Sambrook et al., supra, and Ausubel et al., supra.

Alternatively, the human homologue can be isolated using the CloneCapture cDNA selection Kit (Clontech, Palo Alto, Calif.): a RecA-based system for the rapid enrichment and isolation of cDNA clones of interest without library screening.

### Example 3

Expression of P00210_D09 in E. coli

The P00210_D09 DNA is initially amplified using selected PCR primers. The primers should contain restriction enzyme sites that correspond to the restriction enzyme sites on the selected expression vector. A variety of expression vectors may be employed. An example of a suitable vector is pBR322 (derived from E. coli; see Bolivar et al., Gene, 2:95 [1977]) which contains genes for ampicillin and tetracycline resistance, or a pBR322-based vector. Other, commercially available vectors include various pUC vectors and Bluescript M13. The vector is digested with restriction enzyme and dephosphorylated. The PCR amplified sequences are then ligated into the vector. The vector will preferably include sequences that encode an antibiotic resistance gene, a promoter, such as a T7 or tryptophan (trp) promoter, a polyhis leader (including the first six STII codons, polyhis sequence, and enterokinase cleavage site), the P00210_D09 coding region, lambda transcriptional terminator, and an argu gene.

The ligation mixture is then used to transform a selected E. coli strain using the methods described in Sambrook et al., supra. Transformants are identified by their ability to grow on LB plates and antibiotic resistant colonies are then selected. Plasmid DNA can be isolated and confirmed by restriction analysis and DNA sequencing.

Selected clones can be grown overnight in liquid culture medium such as LB broth supplemented with antibiotics. The overnight culture may subsequently be used to inoculate a larger scale culture. The cells are then grown to a desired optical density, during which the expression promoter is turned on.

After culturing the cells for several more hours, the cells can be harvested by centrifugation. The cell pellet obtained by the centrifugation can be solubilized using various agents known in the art, and the solubilized protein can then be purified using a metal chelating column under conditions that allow tight binding of the poly-his tagged protein.

### Example 4

Expression of P00210 D09 in Yeast

A yeast expression vector is constructed either for intracellular production or secretion of the protein encoded by P00210_D09, using an appropriate yeast promoter, such the promoter of 3-phosphoglycerate kinase, or the promoter regions for alcohol oxidase 1 (AOX1, particularly preferred for expression in Pichia), alcohol dehydrogenase 2, or isocytochrome C. For secretion, the P00210_D09 coding sequence is linked, at its 5′-end, to a mammalian or yeast signal (secretory leader) sequence, such as a yeast alpha-factor or invertase secretory signal. Alternatively, a commercially available yeast expression system is used that can be purchased, for example, from Clontech Laboratories, Inc. (Palo Alto, Calif., e.g. pYEX 4T family of vectors for Saccharomyces cerevisiae), Invitrogen (Carlsbad, Calif., e.g. pPICZ series Easy Select Pichia Expression Kit) or Stratagene (La Jolla, Calif., e.g. ESP™ Yeast Protein Expression and Purification System for S. pombe and pESC vectors for S. cerevisiae).

Yeast cells, such as S. cerevisiae AB 110 strain, or P. pastoris GS 115 (NRRL Y-15851); GS190 (NRRL Y-18014) or PPF1 (NRRL Y-18017) are then transformed by known techniques, e.g. by the polyethylene glycol method (Hinnen, Proc. Natl. Acad. Sci. USA 75:1929 [1978]).

The recombinant protein is subsequently isolated and purified by removing the yeast cells from the fermentation medium by centrifugation and then concentrating the medium using selected cartridge filters. The concentrate containing the expressed protein may be further purified using selected column chromatography resins.

### Example 5

Expression of P00210 D09 in Mammalian Host Cells

The P00210_D09 gene are subjected to PCR using primers containing suitable restriction enzyme cleavage sites to allow ligation into a mammalian expression vector such as pCEP4 (Invitrogen). To facilitate the eventual recovery of the expressed protein, it is advisable to use the 3′ PCR primer to extend the open reading frame of the cloned gene to include an affinity purification tag such as poly-His (E. Hochuli et al 1987, J. Chrom. 411, 177-184) or calmodulin binding peptide (Hathaway et al, J. Biol. Chem. 1981, 256(15):8183-9). Recovery of the PCR fragment may be followed by its cleavage at the new flanking restriction sites and ligation into a similarly cleaved pCEP4 preparation. Transformation of bacteria and preparation of plasmids from transformants is followed by verification of the plasmid structure by restriction analysis.

Expression of the P00210_D09 gene can be accomplished by transient expression in 293 human embryonic kidney cells. For use of vectors such as pCEP4 having the EBV viral origin of replication, 293EBNA cells that are permissive for replication can be used. Transfection is accomplished using a lipid transfection reagent such as Lipofectamine Plus (Life Technologies, Rockville, Md.). Endotoxin-free plasmid DNA (100 μg) is added to 200 μl PLUS reagent and 10 ml DMEM-21 serum free media to give Mix A. This is incubated at room temperature for 15 minutes. Mix B is prepared from 400111 Lipofectamine and 10 ml serum-free DMEM-21. The two mixes are then combined and incubated at room temperature for another 15 minutes. An 850 cm² roller bottle containing the cells to be transfected at 70% confluence is rinsed with serum-free media and 100 ml of serum-free DMEM-2 with 15 mM HEPES pH 7.3 and the DNA-lipid transfection mixture is then added. The cells are then placed in a roller unit at 37° C. for 4 hours after which the volume of media is doubled by addition of DMEM-2 with 15 mM HEPES pH 7.3, 5% FBS and the bottle returned to roller unit overnight. Collect conditioned media every 2-3 days for 2-3 collections.

### Example 6

Expression of P00210 D09 in Baculovirus-Infected Insect Cells

Baculovirus-based expression is performed using one of the commercially available baculovirus expression systems such as, for example, from Bac-N-Blue^{m} (Invitrogen), BacPAK^{m} Baculovirus Expression System (Clontech), BAC-TO-BAC™ (Life Technologies), or Bac Vector System™ (Novagen). Viral infection of insect cells (e.g. Spodoptera frugiperda (“Sf9”) cells (ATCC CRL 1711)) and protein expression and purification are performed following manufacturers' instructions, or as described by O'Reilley et al., Baculovirus expression vectors: A Laboratory Manual, Oxford: Oxford University Press (1994). Optionally, the coding region of the P00210_D09 sequence is fused upstream of an epitope tag contained within a baculovirus expression vector, such as a poly-His tag or an immunoglobulin (Ig) tag (like Fc regions of an IgG). The poly-His or Ig tag aids protein purification.

### Example 7

Preparation of Antibodies that Bind the Polypeptide Encoded by P00210 D09

This example illustrates preparation of monoclonal antibodies that specifically bind the polypeptide encoded by P00210_D09.

Techniques for producing the monoclonal antibodies are known in the art and are described, for instance, in Goding, supra. The immunogen may, for example, be purified protein encoded by P00210_D09. Mice, such as Balb/c, are immunized with the immunogen emulsified in a selected adjuvant, for example Freund's adjuvant, and injected subcutaneously or intraperitoneally in an amount from 1-100 micrograms. Approximately 10 to 12 days later, the immunized mice are boosted with additional immunogen emulsified in the selected adjuvant. Thereafter, for several weeks, the mice may get additional boosts. Serum samples may be periodically obtained from the mice by retro-orbital bleeding for testing in ELISA assays to detect antibodies to the polypeptide encoded by P00210_D09.

After a suitable antibody titer has been detected, the animals “positive” for antibodies can be injected with a final intravenous injection of the immunogen. Three to four days later, the mice are sacrificed and the spleen cells are harvested. The spleen cells are then fused to a selected murine myeloma cell line such as P3X63AgU.1, available from ATCC, No. CRL 1597. The fusions generate hybridoma cells which can then be plated in 96 well tissue culture plates containing HAT (hypoxanthine, aminopterin, and thymidine) medium to inhibit proliferation of non-fused cells, myeloma hybrids, and spleen cell hybrids.

The hybridoma cells will be screened in an ELISA for reactivity against the protein encoded by P00210_D09.

The positive hybridoma cells can be injected intraperitoneally into syngeneic Balb/c mice to produce ascites containing the antibodies. Antibodies are purified by ammonium sulfate precipitation, protein A or protein G chromatography or other techniques well known in the art.

### Example 8

Further Animal Models

The biological function of the P00210_D09 gene and the encoded protein are further characterized in various animal models of heart, kidney and inflammatory disorders.

1. In Vivo Model of Cardiac Hypertrophy

Rats with left ventricular hypertrophy (LVH) are produced essentially as described in Schunkert et al., J. Clin. Invest. 86(6):1913-20 (1990). LVH is induced by pressure overload as a result of constriction of the ascending aorta. A stainless steel clip of 0.6-mm internal diameter is placed on the aorta of anesthetized weanling rats. Control animals undergo thoractomy as a sham operation. Animals usually recover from surgery and appear healthy until about 20 weeks when a few animals may be in demise likely due to heart failure, which typically occurs at this point (Schunkert et al., 1990, supra). The animals are sacrificed and hearts examined 10 weeks and 20 weeks post-operation. Hypertrophy is evident at both time points as determined by changes in left ventricle weight and thickness. Aortic banded rats and sham operated control animals are sacrificed and measured for heart weight, left ventricle (LV) weight, left ventricle thickness, and LV weight/body weight. Usually there are 6 animals per group. Data are expressed as average with standard deviation.

LVH rats are also examined for expression of ANP, BNP, cardiac a-actin, and/or α-myosin heavy chain mRNA, using Northern blot. Levels of these messages are expected to be elevated in the diseased animals, confirming that the banded rats were pressure overloaded and responded with cardiac hypertrophy. Poly A+ mRNA is prepared from each of the animals for assessment of differentially expressed genes in the disease state, using microarray analysis in a preferred embodiment.

2. In vivo Model of Viral Myocarditis

CVB3 infection in mice results in myocardial disease progression, which can be used as a model for examination of the pathogenesis of virus-induced human myocarditis. The virus is directly injurious to myocardial cells early following infection during the preinflammatory period as determined by light and electron microscopic cytological assessment (Arola et al., J. Med. Virol. 47: 251-259 [1995]; Chow et al., Lab. Invest. 64: 55-64 [1991]; McManus et al., Clin. Immunol. Immunopathol. 68:159-169 [1993]; Melnick et al., J. Expert. Med. 93: 247-266 [1951]). Beginning by day two post-infection cytopathic lesions are evident in ventricular myocytes, characterized by cell vacuolar changes, contraction bands and coagulation necrosis (McManus et al., supra). By day 5 post-infection this myocardial injury becomes obscured by inflammatory infiltrates, cellular calcification, and tissue edema.

In a typical protocol, A/J (H-2^{a}) mice (Jackson Laboratories, Bar Harbor, Me., 4 weeks of age) are acclimatised for one week prior to the onset of the experiment. Any mice that dies naturally during the course of the disease are not included in groups of mice to be used for RNA extraction. Mice are euthanized by CO₂ narcosis.

Myocarditic CVB3 (Dr. Charles J. Gauntt; University of Texas, San Antonio, Tex.) is stored at −80° C. Virus is propagated in HeLa cells (American Type Tissue Culture Collection, Rockville, Md.) and is routinely titred before the onset of all experiments using the plaque assay method, with modifications as previously described (Anderson et al., J. Virol. 70: 4632-4645 [1996]).

Adolescent A/J mice are infected with 1×10⁵ pfu of myocarditic CVB3 or PBS sham and euthanized on days 3, 9, and 30 post-infection. Ten to fifteen mice per group (CVB3 infected or sham injected) per time-point (days 3, 9, and 30) are euthanized and heart muscle is removed. Following a wash in sterile phosphate buffered saline, a small portion of the apex of the heart is removed and fixed in 4% paraformaldehyde. The remainder of the heart is flash frozen in liquid nitrogen and stored at −80° C. for future RNA isolation.

Sections from the heart are fixed in fresh DPBS-buffered 4% paraformaldehyde overnight at 4° C. Fixed tissue is dehydrated in graded alcohols, cleared in xylene, embedded in paraffin, and sectioned for hematoxylin and eosin, and Masson's trichrome stains. Serial sections are also prepared for in situ hybridization and nick-end labelling stained. The extent and severity of virus-induced injury (including coagulation necrosis, contraction band necrosis, and cytopathic effects), inflammation, and tissue fibrosis and calcification are evaluated and scored as previously described (Chow et al., supra).

In situ hybridization for CVB3 viral RNA localization is carried out as previously described (Anderson et al., supra; Hohenadl et al., Mol. Cell. Probes 5: 11-20 [1991]). Briefly, tissue sections are incubated overnight in hybridization mixture containing digoxigenin-labelled, CVB3 strand-specific riboprobes. Post-hybridization washing is followed by blocking with 2% normal lamb serum. A sheep anti-digoxigenin polyclonal antibody conjugated to alkaline phosphatase (Boehringer Mannheim PQ, Laval, Canada) is developed in Sigma-Fast nitroblue tetrazolium-BCIP [5-bromo-4-chloro-3-indolylphosphate tuluidinium] (Sigma Chemical Co.). The slides are counterstained in fresh carmalum and examined for reaction product by light microscopy. Poly A+ mRNA is prepared from each of the animals, as described herein, for assessment of differentially expressed genes in the disease states, using microarray.

3. In Vivo Model of Kidney Disease

In yet another representative example, an in vivo model of kidney disease is used to further characterize the differentially expressed genes of the present invention. For example, a rat model of an inherited form of autosomal dominant polycystic kidney disease (ADPKD) can be used, which develops in Han: SPRD rats (Kaspareit-Rittinghaus et al., Transplant Proc. 6: 2582-3 [1990]; Cowley et al., Kidney Int. 43:522-34 [1993]). Renal cysts and renal failure is evident in six months old male heterozygous rats (Cy/+), whereas control rats (+/+) show no sign of cysts or renal failure. Diseased (Cy/+) and normal (+/+) animals are sacrificed and the kidneys removed. For cDNA microarray analysis, poly A+ mRNA is prepared, as described previously, for assessment of differentially expressed genes in the disease state, using microarray analysis in a preferred embodiment.

### Example 9

Cell Culture and RNA Isolation

Primary cultures of rat neonatal cardiac myocytes isolated from the ventricles of 1-2 day old rat pups by trypsin digestion essentially as described (Dunnmon et al., J. Mol. Cell. Cardiol. 22:901-910 [1990]) and plated onto fibronectin coated plates (Becton Dickinson, Bedford, Mass.) in plating media (DMEM21/COON's F12 supplemented with 10% fetal bovine serum) and penicillin and streptomycin). Following cell attachment (˜16-18 h) the plating media was replaced with serum-free media (DMEM21/COON's F12 supplemented with 1 mg/ml bovine serum albumin, penicillin and streptomycin). All experiments were performed following a 24-hour period of serum starvation. The following factors were added to cultures for 2 and 24 hours treatment times: CT-I (cardiotropin-1, 1 ng/ml), Phe (phenylephrine, 101M), Ang II (Angiotensin II, 10 ng/ml), Eth-1 (endothelin 1, 10 ng/ml), TGFβ (transforming factor beta, 10 ng/ml), TNFa (tumor necrosis factor alpha 10 ng/ml), IL-1β (interleukin-1β, 10 ng/ml). Cell culture supernatants were removed for analysis and total RNA was isolated from the cell monolayers using the RNeasy isolation protocol from Qiagen (Valencia, Calif.).

Total RNA for quantitative real-time PCR assays of rodent tissue distribution was obtained from Clontech (Multiple Tissue cDNA Panel K1429-1).

Quantitative Real-Time PCR

Total RNA was analyzed by quantitative real time PCT (Gibson et al., Genome Res. 6:995-1001 [1996]) using an ABI Prism™ 7700 Sequence Detection System (PE Applied Biosystems Foster Ciry, Calif.). This system is based on the ability of the 5′ nuclease activity of Taq polymerase to cleave a nonextendable dual-labeled fluorogenic hybridization probe during the extension phase of PCR. The probe is labeled with reported fluorescent dye at the 5′ end and a quencher fluorescent dye (6-carboxy-tetramethyl-rhodamine) at the 3′ end. When the probe is intact, reporter emission is quenced by the physical proximity of the reporter and quencher fluorescent dyes. Hoever, during the extension phase of PCR, the nucleolytic acivity of the DNA polymerase cleaves the hybridization probe and releases the reporter dye from the probe with a concomitant increase in reporter fluorescence.

Sequence specific primers and probes for rat P00188_D12 and 18S ribosomal RNA were designed using Primer Express software (PE Applied Biosystms, Foster City, Calif.). Fpr P00188_D09 the following forward, reverse and probe primers were synthesized”

(SEQ IS NO:13)

5′-TGGCCTTCGTCTTCGATGTC-3′

(SEQ IS NO:14)

5′-GCCGTCGATCACCTGCAT-3′

(SEQ IS NO:15)

5′-6FAM-CCGGCTCCATGTGGGACGATCT-TAMRA-3′

For 18S ribosomal RNA the following forward, reverse and probe primers were synthesized:

(SEQ IS NO:16)

5′-CGGCTACCACATCCAAGGAA-3′

(SEQ IS NO:17)

5′-GCTGGAATTACCGCGGCT-3′

(SEQ IS NO:18)

5′-6FAM-TGCTGGCACCAGACTTGCCTC-TAMRA-3′

Primers were used at a concentration of 200 nM and probes at l OONM in each reaction. Multiscribe reverse transcriptase and AmpliTaq Gold polymerase. (PE Applied Biosystems, Foster City Calif.) were used in all RT-PCR reactions. RT-PCR parameters were as follows: 48° C. for 30 minutes (reverse transcription), 95C for 10 minutes (AmpliTaq Gold activation) and 40 cycles of 95C for 15 seconds, 60C for 1 minute. Relative threshold cycle numbers for each sample fitted to a five point standard curve (ABI Prism 7700 User Bulletin #2, PE Applied Biosystems, Foster City Calif.). Expression levels were normalized to 18S ribosomal RNA.

Ventricular hypertrophy is initially a compensatory mechanism in which the heart attempts to counteract the effects of pressue overload. Such an overload can be generated by a variety of physiological stimuli. If the transition to decompensated hypertrophy occurs, the progression to a terminal heart failure phenotype often rapidly follows (Chien et al. FASEB J. 5:3037-3046 (1991)). Thus there is great interest in trying to understand the mechanisms that induce and control ventricular hypertrophy.

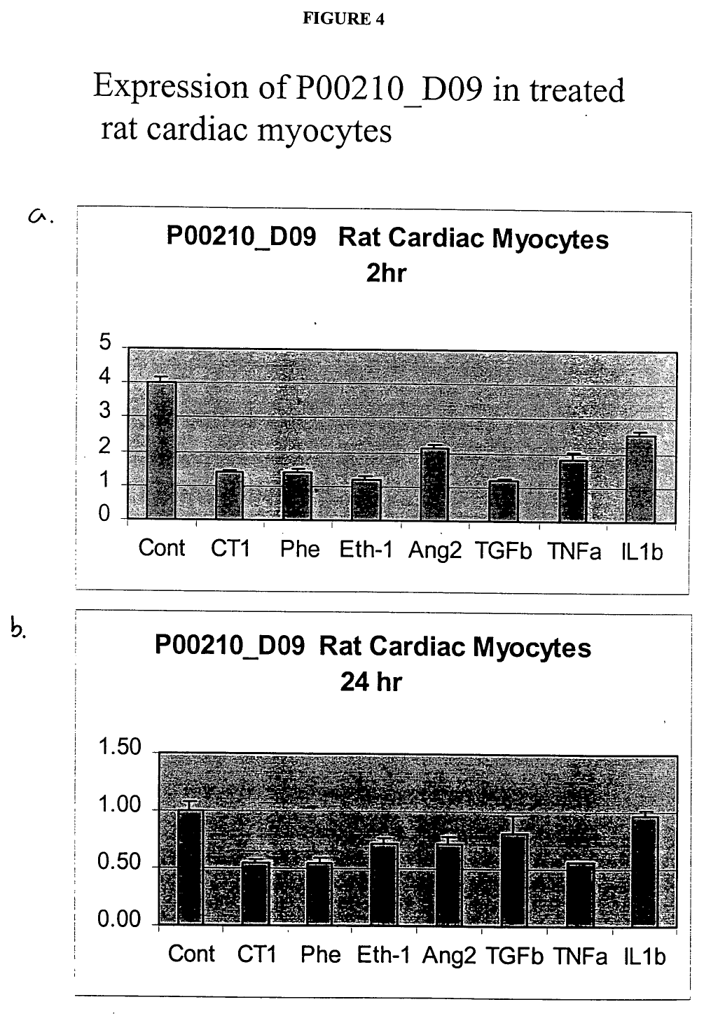

To investigate what factors mediate POO 188_D 12 expression, rat cardiac myocytes were treated with various growth factors and cytokines known to induce cardiac hypertrophy. (FIG. 4).

Treatment of rat cardiac myocytes with CT-1, Phe, Eth-1, Ang2, TGF-beta, and TNF-alpha for 2 hours decreased expression of P00210_D09 mRNA 2 to 3-fold (FIG. 4A). Treatment with CT-1, Phe and TNF-alpha for 24 hours decreased expression of P00210_D09 mRNA 1.8 fold (FIG. 4B). These results suggest that P00210_D09 is a downstream mediator of known factors that induce cardiac hypertrophy. Thus P00210_D09 mRNA may contribute to cardiac hypertrophy and heart failure associated with human disease.

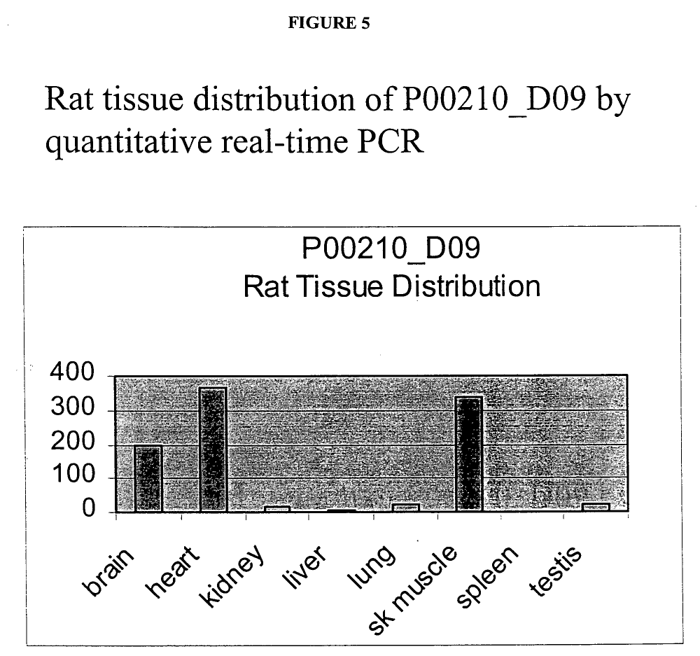

Northern blot analysis of P00210_D09 mRNA indicates that it encodes a rare, heart-specific message (see FIG. 3). To confirm and expand this expression profile, expression of P00210_D09 mRNA was assayed invarious rat tissues by quantitative real-time PCT (FIG. 5). The results verify the predominate expression of P00210_D09 in the heart. Significant levels of P00210_D09 mRNA were also revealed in the brain and skeletal muscle.

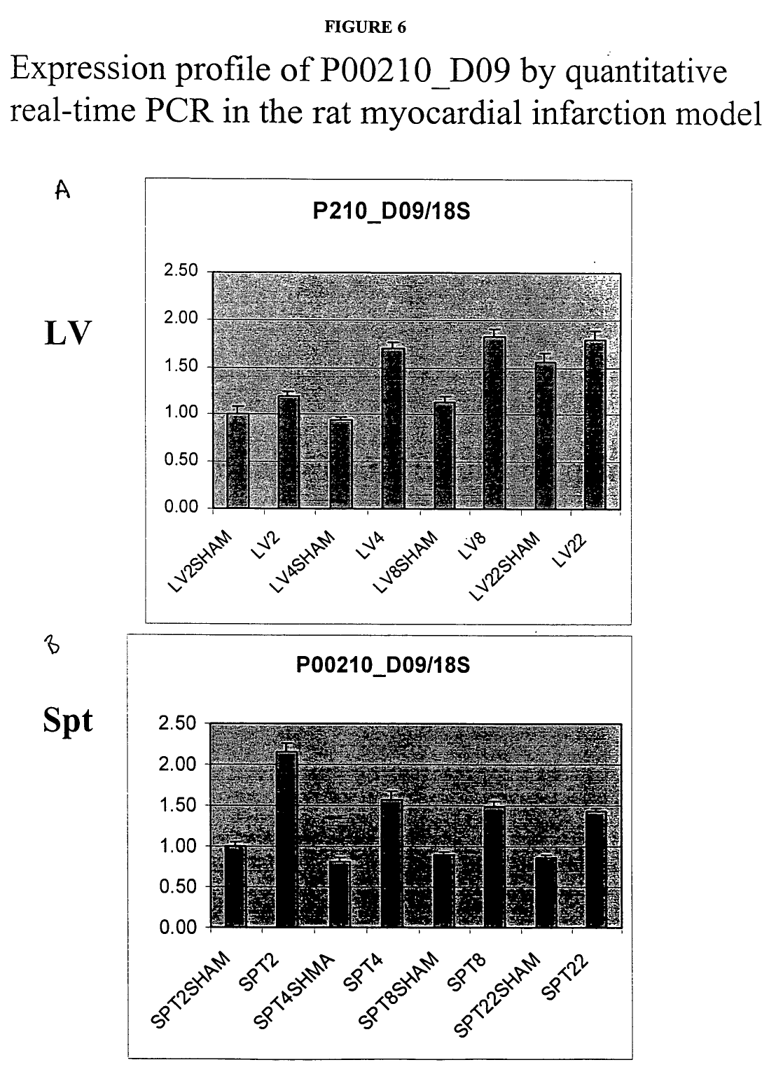

Microarray analysis of the rat myocardial infarction model suggested a possible overexpression of P00210_D09 at 12 weeks in the left ventricle (see Example 1). To expand and confirm these results, expression of P00210_D09 mRNA was assayed in this model by real-time quantitative PCR (FIG. 6). Following surgically induced myocardial infarction or a sham operation (SHAM), significant induction of P00210_D09 mRNA expression was observed at 4 weeks (LV4) and 8 weeks (LV8) in the left ventricle (FIG. 6A) and at 2 weeks (Spt2) and 4 weeks (Spt4) in the septum (FIG. 6B).

All references cited throughout the specification, including the examples, are hereby expressly incorporated by reference.

1. A method for identifying genes differentially expressed in a disease state, comprising:
providing a subject;
establishing a normal gene expression pattern for the subject;
inducing the disease state in the subject, wherein the disease state is selected from the group consisting of a cardiac disease, a renal disease, and an inflammatory disease;
establishing a disease state expression pattern for the subject; and
determining the relative expression of a gene in the subject suffering from the disease state as compared to the normal gene expression pattern; wherein the gene comprises SEQ ID NO: 2.

2. The method of claim 1, wherein the gene encodes a protein of SEQ ID NO:1.

3. The method of claim 1, wherein expression of the gene increases in the disease state.

4. The method of claim 1, wherein expression of the gene decreases in the disease state.

5. The method of claim 1, wherein the cardiac disease is selected from the group consisting of congestive heart failure, myocardial infarction, and viral myocarditis.

6. The method of claim 1, wherein the renal disease is acute renal failure.

7. A method for identifying candidate compounds that modify expression a gene differentially expressed in a disease state, comprising:
inducing the disease state in a subject, wherein the disease state is selected from the group consisting of cardiac disease, renal disease, and inflammatory disease;
establishing a disease state expression pattern for the subject;
providing a candidate compound to the subject in the disease state; and
identifying the candidate compound that modifies expression of a gene in the subject;
wherein the gene comprises SEQ ID NO: 2.

8. The method of claim 7, wherein the candidate compound increases expression of the gene encoding SEQ ID: 2.

9. The method of claim 7, wherein the candidate compound decreases expression of the gene encoding SEQ ID: 2.

10. The method of claim 7, wherein the gene encodes a protein of SEQ ID NO:1.

11. The method of claim 7, wherein expression of the gene increases in the disease state.

12. The method of claim 7, wherein expression of the gene decreases in the disease state.

13. The method of claim 7, wherein the cardiac disease is selected from the group consisting of congestive heart failure, myocardial infarction, and viral myocarditis.

14. The method of claim 7, wherein the renal disease is acute renal failure.
